(19) United States
(12) Patent Application Publication
(10) Pub. No.: US 2005/0158371 A1
(43) Pub. Date: Jul. 21, 2005

(54) Novel external agent

(75) Inventors: Fumio Nishikado, Osaka (JP); Shuzo Tagashira, Osaka (JP); Koichi Saito, Osaka (JP); Toshiyuki Hosokawa, Osaka (JP)

Correspondence Address: BIRCH STEWART KOLASCH & BIRCH, PO BOX 747, FALLS CHURCH, VA, 22040-0747, US

(73) Assignee: Sumitomo Pharmaceuticals Co., Ltd., Osaka-Shi (JP)

(21) Appl. No.: 10/921,960

(22) Filed: Aug. 20, 2004

Related U.S. Application Data
(63) Continuation-in-part of application No. PCT/JP03/01409, filed on Feb. 12, 2003.
(63) Continuation-in-part of application No. PCT/JP04/11385, filed on Aug. 2, 2004.

(30) Foreign Application Priority Data
Feb. 12, 2002 (JP) 33975/2002
Jul. 29, 2002 (JP) 219047/2002
Aug. 5, 2003 (JP) 286418/2003
Aug. 7, 2003 (JP) 289457/2003

(51) Int. Cl. A61K 31/42; A61K 9/70

(52) U.S. Cl. 424/449; 514/378

(57) ABSTRACT

The present invention relates to a transdermal administration preparation for external application such as ointment, cream and the like, which contains SMP-114 or leflunomide or a pharmaceutically acceptable acid addition salt thereof as an active ingredient. The present invention further relates to a pharmaceutical composition for transdermal administration which contains, a) as an active ingredient, N-(4-trifluoromethylphenyl)-5-methylisoxazole-4-carboxamide or an active motabolite thereof or a pharmaceutically acceptable salt thereof; and B)(1) a carrier for transdermal administration which contains a base for dissolution in a proportion of not less than 40 w/w %, or (2) a carrier for transdermal administration which contains a hydrophobic base for suspension having no polar group in a molecule in a proportion of not less than 70 w/w %. According to the present invention, a novel means of transdermal administration of SMP-114 or leflunomide or an active motabolite thereof or a pharmaceutically acceptable acid addition salt thereof can be provided.

### TECHNICAL FIELD OF THE INVENTION

The present invention relates to a dosage form for external application comprising an isoxazole derivative having two substituents as an active ingredient. More particularly, the present invention relates to a dosage form for external application comprising SMP-114 or a pharmaceutically acceptable acid addition salt thereof as an active ingredient. Furthermore, the present invention relates to a dosage form for external application comprising N-(4-trifluoromethylphenyl)-5-methylisoxazole-4-carboxamide (hereinafter sometimes to be abbreviated as leflunomide) as an active ingredient. More specifically, the present invention relates to a pharmaceutical composition for transdermal administration, which is used for directly treating inflammatory lesion by transdermally administering SMP-114 or leflunomide, which is an isoxazole derivative, or a pharmaceutically acceptable acid addition salt thereof.

Moreover, the present invention relates to a pharmaceutical composition for transdermal administration, which comprises, as an active ingredient, leflunomide or an active metabolite thereof, or a pharmaceutically acceptable salt thereof. More particularly, the present invention relates to a pharmaceutical composition for transdermal administration for the treatment of an inflammatory lesion and the like, which comprises leflunomide, or N-(4-trifluoromethylpheyl)-2-cyano-3-hydroxy-crotonamide, which is an active metabolite thereof, or a pharmaceutically acceptable salt thereof in (1) a carrier for transdermal administration, which contains a particular base for dissolution, in a dissolution state, or in (2) a carrier for transdermal administration, which contains a particular base for suspension, in a suspension state, and an administration method thereof.

### BACKGROUND OF THE INVENTION

SMP-114 is 3-[(1S)-1-(2-fluorobiphenyl-4-yl)ethyl]-5-{[amino(morpholin-4-yl)methylene]amino}isoxazole, which is a compound represented by the following structural formula. The production method thereof and the like are described in US 6100260 and WO 98/47880.

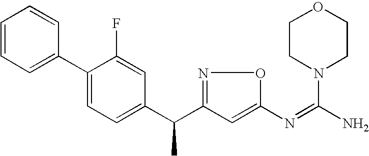

Leflunomide is N-(4′-trifluoromethylphenyl)-5-methylisoxazole-4-carboxamide, which is a compound represented by the following structural formula. The production method thereof and the like are described in JP-A-55-83767, U.S. Pat. No. 4,087,535, EP 13376, U.S. Pat. No. 4,284,786, U.S. Pat. No. 4,351,841 and the like.

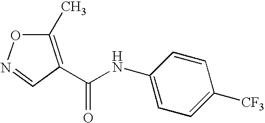

N-(4-Trifluoromethylpheyl)-2-cyano-3-hydroxy-crotonamide is a compound represented by the following structural formula, which has an antiinflammatory activity and an analgetic activity. It is known as an active metabolite of leflunomide and the detail thereof is described in U.S. Pat. No. 4,061,767.

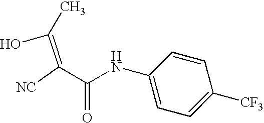

JP-B-3285226 describes pharmaceuticals for the treatment of hyperacute or chronic rejection reaction against a transplanted organ in an organ recipient, which contain leflunomide or N-(4-trifluoromethylpheyl)-2-cyano-3-hydroxy-crotonamide (or a pharmaceutically acceptable salt thereof).

In addition, JP-B-3131693 describes an ophthalmic composition for the treatment of uveitis, retinitis, allergy, dry eye, which contains 5-methyl-isoxazole-4-carboxanilide or 2-hydroxyethylidene-cyanoacetanilides.

JP-B-2930281 describes a pharmaceutical agent for the prophylaxis or treatment of cutaneous diseases such as psoriasis, atopic dermatitis, allergic dermatitis, dermatitis medicamentosa and the like, which contains, as an active ingredient, a particular compound containing leflunomide or N-(4-trifluoromethylpheyl)-2-cyano-3-hydroxy-crotonamide.

In general, since transdermal administration permits direct administration of a drug to a lesion, it affords advantages in that the drug concentration at the lesion can be topical increased, side effects are expected to be decreased by avoiding a liver first-pass effect of oral administration, administration frequency can be reduced and sustention of efficacy can be afforded. Moreover, it is also advantageous for patients who find oral administration to be difficult, because administration is easy. Therefore, various pharmaceutical agents for transdermal administration have been studied.

However, researches of external agent of SMP-114 or leflunomide which is an isoxazole derivative, as in the present invention, has not been reported, and therefore, researches of antiinflammatory agent and antirheumatic agent in the form of an external agent of the present invention has not been reported at all.

SMP-114 is reported to show potent antiinflammatory activity and antirheumatic activity upon oral absorption. However, there is no report on SMP-114 showing a direct effect on the lesion by transdermal absorption, and what preparation is transdermally absorbable cannot be even predicted.

Leflunomide is known to be absorbed by oral administration, metabolized in blood, reaches the lesion in the form of an active metabolite, where it exhibits a treatment effect.

However, no report has been found that mainly aims at an agent for transdermal administration of leflunomide or N-(4-trifluoromethylpheyl)-2-cyano-3-hydroxy-crotonamide, which is an active metabolite thereof (or a pharmaceutically acceptable salt thereof), for the treatment of diseases other than dermatosis such as chronic rheumatism, arthritis and the like. Moreover, it was not possible to assume that leflunomide or an active metabolite thereof, directly administered transdermally to the lesion of inflammation or rheumatism, exhibits a treatment effect on the topical lesion. Furthermore, an antirheumatic effect and anti-inflammatory effect afforded by these drugs upon transdermal administration using a particular carrier have not been reported.

### SUMMARY OF THE INVENTION

The problem to be solved by the present invention is provision of a novel administration means of SMP-114 or leflunomide, which is an isoxazole derivative having two substituents. Another problem to be solved by the present invention is provision of a novel pharmaceutical composition for transdermal administration containing leflunomide or an active metabolite thereof, or a pharmaceutically acceptable salt thereof as an active ingredient, which has a superior treatment effect and stability.

The present inventors have conducted intensive studies in an attempt to solve the aforementioned problems, and found that, by preparing an isoxazole derivative having two substituents, such as SMP-114, leflunomide and the like, or a pharmaceutically acceptable acid addition salt thereof into a dosage form for external application, each pharmaceutical is taken into living organism, and as a result, an antiinflammatory or antirheumatic effect can be exhibited in the lesion.

The present inventors have further found that a pharmaceutical composition for transdermal administration, which contains leflunomide as an active ingredient, wherein the active ingredient is contained in a carrier for transdermal administration, which contains a particular base,
(1) is superior in stability,
(2) shows enhanced uptake of the pharmaceutical agent into a living body at the administration site, and
(3) shows markedly improved antirheumatic effect or anti-inflammatory effect at the lesion.
   Further studies resulted in the completion of the present invention.

Accordingly, the present invention provides the following.
[1] A dosage form for external application comprising an isoxazole derivative having two substituents as an active ingredient.
[2] The dosage form for external application of the above-mentioned [1], where the isoxazole derivative is an oral antiinflammatory drug or an oral antirheumatic drug.
[3] The dosage form for external application of the above-mentioned [1] or [2], which is an ointment or cream.
[4] The dosage form for external application of the above-mentioned [1] or [2], which is a solution.
[5] The dosage form for external application of any of the above-mentioned [1]-[4], wherein the isoxazole derivative is SMP-114 or a pharmaceutically acceptable acid addition salt thereof.
[6] The dosage form for external application of any of the above-mentioned [1]-[4], wherein the isoxazole derivative is leflunomide.
[7] The dosage form for external application of any of the above-mentioned [5]-[6], wherein an amount of SMP-114 or leflunomide is 0.1-10 w/w %.
[8] A pharmaceutical composition for transdermal administration, which comprises SMP-114 or leflunomide or a pharmaceutically acceptable acid addition salt thereof as an active ingredient.
[9] The pharmaceutical composition of the above-mentioned [8], which is in the form of an ointments, creams, lotions, solutions, suspensions, gel preparations or plasters or transdermal patch.
[10] Use of SMP-114 or leflunomide for the manufacture of a therapeutic agent for an inflammation or a rheumatism by transdermal administration, which comprises a pharmaceutical composition of the above-mentioned [8] or [9].
[11] A transdermal therapeutic agent for an inflammation or a rheumatic disease, which comprises SMP-114 or leflunomide as an active ingredient.
[12] The transdermal therapeutic agent of the above-mentioned [11], wherein an amount of SMP-114 or leflunomide is 0.1-10 w/w %.
[13] The dosage form for external application of the above-mentioned [5] or [6], wherein an amount of SMP-114 or leflunomide is 0.2-5 w/w %.
[14] The dosage form for external application of the above-mentioned [5] or [6], wherein an amount of SMP-114 or leflunomide is 1-5 w/w %.
[15] The pharmaceutical composition of the above-mentioned [8] or [9], wherein an amount of SMP-114 or leflunomide is 0.1-10 w/w %.
[16] The pharmaceutical composition of the above-mentioned [8] or [9], wherein an amount of SMP-114 or leflunomide is 0.2-5 w/w %.
[17] The pharmaceutical composition of the above-mentioned [8] or [9], wherein an amount of SMP-114 or leflunomide is 1-5 w/w %.
[18] The use of the above-mentioned [10], wherein an amount of SMP-114 or leflunomide in the pharmaceutical composition for transdermal administration is 0.1-10 w/w %.
[19] The use of the above-mentioned [10], wherein an amount of SMP-114 or leflunomide in the pharmaceutical composition for transdermal administration is 0.2-5 w/w %.
[20] The use of the above-mentioned [10], wherein an amount of SMP-114 or leflunomide in the pharmaceutical composition for transdermal administration is 1-5 w/w %.
[21] The transdermal therapeutic agent of the above-mentioned [11], wherein an amount of SMP-114 or leflunomide is 0.2-5 w/w %.
[22] The transdermal therapeutic agent of the above-mentioned [11], wherein an amount of SMP-114 or leflunomide is 1-5 w/w %,
[23] A commercial package comprising a pharmaceutical composition of any of the above-mentioned [8], [9] and [15]-[17] and a written matter associated therewith, the written matter stating that the pharmaceutical composition can or should be used transdermally for treating an inflammation or a rheumatism.
[24] A method of treating inflammation or a rheumatism, which comprises transdermally administering to an animal an effective amount of a pharmaceutical composition for transdermal administration of any of the above-mentioned [8], [9] and [15]-[17].
[25] A pharmaceutical composition for transdermal administration, which comprises a) leflunomide or an active metabolite thereof, or a pharmaceutically acceptable salt thereof as an active ingredient; and
b)(1) a carrier for transdermal administration, which comprises a base for dissolution in a proportion of not less than 40 w/w %, or
(2) a carrier for transdermal administration, which comprises a hydrophobic base for suspension having no polar group in a molecule in a proportion of not less than 70 w/w %.
[26] The pharmaceutical composition of the above-mentioned [25], wherein the active metabolite is N-(4-trifluoromethylpheyl)-2-cyano-3-hydroxy-crotonamide.
[27] The pharmaceutical composition of the above-mentioned [25] or [26], which is an active ingredient dissolution type composition, wherein the carrier for transdermal administration comprises a base for dissolution in a proportion of not less than 40 w/w %.
[28] The pharmaceutical composition of the above-mentioned [27], wherein the base for dissolution is one kind of base or a mixture of two or more kinds thereof selected from dibasic acid dialkyl esters, polyoxyethylene polyoxypropylene glycols, medium chain fatty acid triglycerides and macrogols.
[29] The pharmaceutical composition of the above-mentioned [27], wherein the carrier for transdermal administration has a hydrocarbon oil content of not more than 40 w/w %.
[30] The pharmaceutical composition of the above-mentioned [27], wherein the carrier for transdermal administration has a content of the base for dissolution of not less than 50 w/w %.
[31] The pharmaceutical composition of the above-mentioned [27], wherein the carrier for transdermal administration further comprises a lipophilic nonionic surfactant.
[32] The pharmaceutical composition of the above-mentioned [28], wherein the dibasic acid dialkyl ester is one kind of said ester or a mixture of two or more kinds thereof selected from diethyl sebacate, diisopropyl sebacate and diisopropyl adipate.
[33] The pharmaceutical composition of the above-mentioned [28], wherein the polyoxyethylene polyoxypropylene glycol is one kind of said glycol or a mixture of two or more kinds thereof selected from those that are liquid at 30° C.
[34] The pharmaceutical composition of the above-mentioned [28], wherein the medium chain fatty acid triglyceride is one kind of said triglyceride or a mixture of two or more kinds thereof selected from triglycerides comprising a fatty acid having 8 to 10 carbon atoms.
[35] The pharmaceutical composition of the above-mentioned [31], wherein the lipophilic nonionic surfactant is α-monoalkyl glyceryl ether.
[36] The pharmaceutical composition of the above-mentioned [31], wherein the carrier for transdermal administration has a lipophilic nonionic surfactant content of 0.1-10 w/w %.
[37] The pharmaceutical composition of the above-mentioned [27], which has a dosage form of a solution, an ointment, a gel preparation or a plaster.
[38] The pharmaceutical composition of the above-mentioned [27], which has a dosage form of a solution, an ointment or a gel preparation.
[39] The pharmaceutical composition of the above-mentioned [25] or [26], wherein the carrier for transdermal administration comprises a hydrophobic base for suspension having no polar group in a molecule in a proportion of not less than 70 w/w %, and the composition comprises the active ingredient stably suspended in the carrier.
[40] The pharmaceutical composition of the above-mentioned [39], wherein the hydrophobic base for suspension is a hydrocarbon oil.
[41] The pharmaceutical composition of the above-mentioned [39], wherein the hydrophobic base for suspension is a hydrocarbon gel.
[42] The pharmaceutical composition of any of the above-mentioned [39]-[41], wherein the carrier for transdermal administration consists only of a hydrophobic base for suspension having no polar group in a molecule.
[43] The pharmaceutical composition of the above-mentioned [39], wherein all the active ingredient particles suspended in the carrier for transdermal administration substantively have a particle size of not more than 100 μm and the average particle size is not more than 20 μm.
[44] The pharmaceutical composition of the above-mentioned [39], wherein all the active ingredient particles suspended in the carrier for transdermal administration substantively have a particle size of not more than 20 μm and the average particle size is not more than 10 μm.
[45] The pharmaceutical composition of the above-mentioned [39], which has a dosage form of an ointment, a liquid or a plaster.
[46] The pharmaceutical composition of the above-mentioned [45], which has a dosage form of a suspended ointment or a suspended liquid.
[47] The pharmaceutical composition of the above-mentioned [25], which comprises the active ingredient in a proportion of 0.1-10 w/w %.
[48] Use of leflunomide or an active metabolite thereof, or a pharmaceutically acceptable salt thereof for the manufacture of a transdermally administered therapeutic agent for chronic rheumatism or arthritis, which comprises a composition for transdermal administration of the above-mentioned [25].
[49] An administration method for the treatment of chronic rheumatism or arthritis, which comprises transdermally administering a pharmaceutical composition for transdermal administration, which comprises
a) leflunomide or an active metabolite thereof, or a pharmaceutically acceptable salt thereof as an active ingredient; and
b)(1) a carrier for transdermal administration, which comprises a base for dissolution in a proportion of not less than 40 w/w %, or
(2) a carrier for transdermal administration, which comprises a hydrophobic base for suspension having no polar group in a molecule in a proportion of not less than 70 w/w %.
[50] The administration method of the above-mentioned [49], wherein the carrier for transdermal administration comprises a base for dissolution in a proportion of not less than 40 w/w %, and the active ingredient is transdermally administered in a dissolution state in the carrier for transdermal administration.

The pharmaceutical composition for transdermal administration and the like of the present invention show an anti-inflammatory effect and an antirheumatic effect by administration in a dosage form for external application directly to the skin near the lesion. In addition, the soluble-type pharmaceutical composition for transdermal administration and the suspended-type pharmaceutical composition for transdermal administration of the present invention, which contain leflunomides, stably retain leflunomides and are superior in preparation stability.

### BRIEF DESCRIPTION OF THE DRAWINGS

FIG. 1 is a graph showing a treatment effect of SMP-114 ointment on adjuvant arthritis in Experimental Example 2A.

FIG. 2 is a graph showing a treatment effect of SMP-114 suspension/solution on adjuvant arthritis in Experimental Example 3A.

FIG. 3 is a graph showing a treatment effect of a leflunomide suspension on adjuvant arthritis in Experimental Example 4A.

FIG. 4 is a graph showing a treatment effect of a leflunomide ointment on adjuvant arthritis in Experimental Example 5A.

FIG. 5 is a graph showing a treatment effect of a dosage form for external application of leflunomide on a non-injected foot having adjuvant arthritis in Experimental Example 1B.

FIG. 6 is a graph showing a treatment effect of a dosage form for external application of leflunomide on an injected foot having adjuvant arthritis in Experimental Example 1B.

FIG. 7 is a graph showing a treatment effect of a dosage form for external application of leflunomide on a non-injected foot having adjuvant arthritis in Experimental Example 2B.

FIG. 8 is a graph showing a treatment effect of a dosage form for external application of leflunomide on an injected foot having adjuvant arthritis in Experimental Example 2B.

FIG. 9 is a graph showing a treatment effect of a dosage form for external application of leflunomide on a non-injected foot having adjuvant arthritis in Experimental Example 3B.

FIG. 10 is a graph showing a treatment effect of a dosage form for external application of leflunomide on an injected foot having adjuvant arthritis in Experimental Example 3B.

FIG. 11 is a graph showing an effect of a dosage form for external application of leflunomide on body weight change in adjuvant arthritis in Experimental Example 3B.

FIG. 12 is a graph showing a treatment effect of a dosage form for external application of leflunomide on a non-injected foot having adjuvant arthritis in Experimental Example 4B.

FIG. 13 is a graph showing a treatment effect of a dosage form for external application of leflunomide on an injected foot having adjuvant arthritis in Experimental Example 4B.

FIG. 14 is a graph showing a shift in the serum concentration when a dosage form for external application of leflunomide is transdermally administered in Experimental Example 5B.

### DETAILED DESCRIPTION OF THE INVENTION

The present invention is explained in detail in the following.

The “isoxazole derivative having two substituents” of the present invention refers to a compound wherein two positions of an isoxazole ring are substituted by a substituent other than hydrogen. As such di-substituted isoxazole derivative, those described in conventional textbooks of organic chemistry or the manual of Beilstein and the like can be mentioned. As preferable “isoxazole derivative having two substituents”, for example, SMP-114 and N-(4-trifluorommethylphenyl)-5-methylisoxazole-4-carboxamide (hereinafter sometimes to be abbreviated as leflunomide in the present specification) can be mentioned.

The “oral antiinflammatory drug or oral antirheumatic drug” of the present invention refers to a di-substituted isoxazole derivative that can be administered orally and that has an antiinflammatory effect or antirheumatic effect. For example, preferred are SMP-114 and leflunomide.

As the pharmaceutically acceptable acid addition salt of SMP-114 or leflunomide, which is an isoxazole derivative, for example, inorganic acid addition salts such as hydrochloride, hydrobromide, sulfate, phosphate, nitrate and the like, organic acid addition salts such as acetate, propionate, succinate, lactate, malate, tartarate, citrate, maleate, fumarate, methanesulfonate, p-toluenesulfonate, ascorbate and the like, and the like can be mentioned.

The active metabolite of leflunomide is N-(4-trifluoromethylpheyl)-2-cyano-3-hydroxy-crotonamide (hereinafter sometimes to be abbreviated as leflunomide metabolite in the present specification). As a pharmaceutically acceptable salt of an active metabolite thereof, for example, alkali metal salts such as sodium, potassium and the like, alkaline earth metal salts such as calcium and the like, ammonia, organic ammonium salts such as triethylamine, pyridine and the like, and the like can be mentioned.

In addition, the present invention includes solvates of isoxazole derivative, such as hydrates or solvates (e.g., alcohol solvates such as ethanol solvate and the like) of SMP-114 or a pharmaceutically acceptable acid addition salt thereof and the like, hydrate, solvate (e.g., alcohol solvates such as ethanol solvate and the like) of leflunomide and an active metabolite thereof, or a pharmaceutically acceptable acid addition salt thereof, and the like.

As the dosage form for “external application containing isoxazole derivative having two substituents as an active ingredient”, “external application containing SMP-114”, “external application containing leflunomide”, “a pharmaceutical composition for transdermal administration, which contains SMP-114 or leflunomide as an active ingredient” and the like of the present invention, a dosage form conventionally used for external application generally, such as ointments, creams, lotions, solutions, suspensions, gel preparations, plasters(including transdermal patch etc.) and the like can be mentioned.

These dosage forms can be manufactured using conventional base, adhesives and the like according to a conventional method. For example, they can be manufactured according to the descriptions in JP-B-2651616 (U.S. Pat. No. 5,164,416), WO96/12465 and the like.

As the ointments, for example, oleaginous ointment, water-soluble ointment and the like can be mentioned, for which a base can be selected according to each object.

Of these, for the oleaginous ointment, a base appropriately selected from those generally known as a base for ointment, such as petrolatum, purified petrolatum, paraffin, liquid paraffin, lanolin, purified lanolin, hydrocarbon, higher alcohols, fatty oils such as vegetable oil, animal oil and the like, fatty acid esters, Plastibase®, glycols, higher fatty acid and the like is used.

Specifically, for example, purified lanolin, lanolin, petrolatum, white petrolatum, yellow beeswax, white beeswax, solid paraffin, microcrystalline wax, hydrogenated oil, glyceryl trimyristate, stearic acid, isostearic acid, stearyl alcohol, cetyl alcohol, glyceryl monostearate, butyl stearate, hexadecyl isostearate, isostearyl palmitate, octyldodecyl myristate, cetyl myristate, liquid lanolin, liquid paraffin, squalane, squalene, polybutenes, isopropyl palmitate, isopropyl myristate, diethyl sebacate, diisopropyl adipate, castor oil, glyceryl tricaprylate-caprate, glyceryl triisooctanoate, oleyl oleate, decyl oleate, oleyl alcohol, silicon oils and the like are used, and one or plural from these bases are appropriately selected according to the object viscosity and used for preparation.

As the water-soluble ointment, for example, macrogols (polyethylene glycols) such as macrogol 200, macrogol 400, macrogol 1500, macrogol 1540, macrogol 4000, macrogol 20000 and the like, alcohols such as glycerin, propylene glycol and the like, water-soluble polymers such as povidone, polyvinyl alcohol and the like, and the like can be used, and, like oleaginous ointment, one or plural from these bases are appropriately selected according to the object viscosity and used for preparation.

Besides the above-mentioned ointments, hydrophilic ointment, hydrophilic petrolatum, simple ointment, white ointment, macrogol ointment, absorptive ointment and the like described in the Japan Pharmacopoeia can be also used as the ointment.

Creams are prepared from an oil phase component appropriately selected from, for example, petrolatum, purified petrolatum, paraffin, liquid paraffin, fatty oils (e.g., vegetable oil, animal oil and the like), fatty acid esters, higher alcohols, lanolin, purified lanolin, glycols, higher fatty acids and the like, and an aqueous phase according to the object viscosity and emulsion type.

Creams are prepared from an oil phase component appropriately selected from, for example, petrolatum, purified petrolatum, paraffin, liquid paraffin, fatty oils (e.g., vegetable oil, animal oil and the like), fatty acid esters, higher alcohols, lanolin, purified lanolin, glycols, higher fatty acids and the like, and an aqueous phase according to the object viscosity and emulsion type.

Of these, as the oil phase component, for example, purified lanolin, lanolin, petrolatum, white petrolatum, yellow beeswax, white beeswax, paraffin, microcrystalline wax, hydrogenated oil, glyceryl trimyristate, stearic acid, isostearic acid, stearyl alcohol, cetyl alcohol, glyceryl monostearate, butyl stearate, hexadecyl isostearate, isostearyl palmitate, octyldodecyl myristate, cetyl myristate, liquid lanolin, liquid paraffin, squalane, squalene, polybutenes, isopropyl palmitate, isopropyl myristate, diethyl sebacate, diisopropyl adipate, castor oil, glyceryl tricaprylate-caprate, glyceryl triisooctanoate, oleyl oleate, decyl oleate, oleyl alcohol, silicon oils and the like can be mentioned, and a mixture of one or more kinds of these oil phase components can be used as an oil phase.

As the aqueous phase, ion exchange water, distilled water and the like, purified water generally used for pharmaceutical products, and in addition to the purified water, for example, macrogols (polyethylene glycols) such as macrogol 200, macrogol 400, macrogol 1500, macrogol 1540, macrogol 4000, macrogol 20000 and the like, alcohols such as glycerin, propylene glycol and the like, water-soluble polymers such as povidone, polyvinyl alcohol and the like, and the like can be also used, which are dissolved in the above-mentioned purified water according to the object and used as an aqueous phase.

For creams, an emulsifier can be used in addition to the above-mentioned oil phase and aqueous phase, and according to the object viscosity and emulsion type, creams are prepared using appropriately determined amounts of an oil phase, an aqueous phase and an emulsifier to be added.

As the lotions, for example, a suspension lotion wherein oxidized zinc, talc and the like are suspended in one or more aqueous components selected from ethanol, glycerin, glycols, water and the like, an emulsion lotion emulsified into an O/W type, and the like can be mentioned.

Of these, in the case of an emulsion lotion, an oil phase component, an aqueous phase, an emulsifier and the like similar to those for the above-mentioned cream can be used, which are appropriately combined according to the object viscosity.

As the suspensions, for example, oleaginous suspension, aqueous suspension and the like can be mentioned, for which a base can be selected according to the object.

Of these, as the oleaginous suspension, for example, liquid paraffin, hydrocarbon, higher alcohols, and the like, as well as liquid oil such as squalane, squalene, octyldodecyl myristate, isopropyl myristate, oleyl oleate, decyl oleate and the like can be used.

As the aqueous suspension, moreover, liquid alcohols such as glycerin and the like can be used.

As the solutions, for example, liquid oil such as diethyl sebacate, diisopropyl adipate, glyceryl tricaprylate-caprate and the like, and where necessary, ethanol, isopropyl alcohol, glycerin, propylene glycol, polyethylene glycols and the like are used as a base.

As the gel preparations, in the case of an oleaginous gel, for example, an oil to be the base is gelatinized with a polymer such as polyethylene and the like and used as a base, and in the case of an aqueous gel, one gelatinized with an aqueous polymer such as carboxymethyl polymer, hydroxypropyl cellulose, polyvinyl alcohol and the like is used as a base.

In addition, the ointments, creams, lotions, solutions, suspensions and gel preparations can contain, as necessary, a thickener, a stabilizer, a humectant (a wetting agent), a preservative, an emulsifier, a suspending agent, a pH adjuster and the like.

As the thickener, a water-soluble polymer having a molecular weight of about 100,000-5,000,000, preferably about 1,000,000-3,000,000, can be mentioned and, for example, carboxyvinyl polymers, hydroxyethyl celluloses, hydroxypropyl celluloses, hydroxypropylmethyl celluloses and the like can be mentioned. Specifically, as the carboxyvinyl polymers, Carbopol 934, 940 or 941 manufactured by B.F. Goodrich, Hiviswako 103, 104 or 105 and the like manufactured by Wako Pure Chemical Industries, Ltd.; as the hydroxypropylmethyl celluloses, TC-5R, Metolose 90SH and the like manufactured by Shin-Etsu Chemical Co., Ltd.; and as the hydroxypropyl celluloses, HPC—H and the like manufactured by Nippon Soda Co, Ltd. can be mentioned.

As the pH adjuster, for example, lactic acid, citric acid, phosphoric acid and the like can be mentioned for adjusting to a lower pH range, and sodium hydroxide, potassium hydroxide, sodium lactate, sodium citrate, monoethanolamine and diisopropanolamine and the like can be mentioned for adjusting to a higher pH range. Addition of carboxyvinyl polymer, which is a water-soluble polymer, can also achieve a lower pH.

As the humectant, propylene glycol, glycerin, 1,3-butylene glycol and the like can be mentioned.

As the stabilizer, ascorbic acid, dibutylhydroxytoluene, sodium thiosulfate, sodium thioglycolate, sodium thiomalate, erythorbic acid, sodium erythorbate, sodium pyrosulfite, benzoic acid, sodium benzoate, sodium alginate, sodium caprylate, L-arginine, L-cysteine, dl-α-tocopherol, tocopherol acetate, propyl gallate, disodium edetate and the like can be mentioned.

As the preservative, benzethonium chloride, benzalkonium chloride, methylparaben, ethylparaben, propylparaben, chlorobutanol, benzyl alcohol, thimerosal and the like can be mentioned.

As the emulsifier, one appropriately selected from those generally used for pharmaceutical products can be used. Specifically, sorbitan fatty acid esters such as sorbitan monostearate, sorbitan monopalmitate, sorbitan monooleate, sorbitan sesquioleate and the like; glycerin fatty acid esters such as glyceryl monostearate, glyceryl monooleate and the like; sucrose fatty acid esters; polysorbates such as polyoxyethylene sorbitan monostearate, polyoxyethylene sorbitan monooleate, polyoxyethylene sorbitan trioleate and the like; polyoxyethylene alkyl ethers such as polyoxyethylene cetyl ether, polyoxyethylene oleyl ether and the like; polyoxyethylene fatty acid esters such as polyoxyl 40 stearate, polyethyleneglycol monolaurate and the like; and the like can be mentioned.

The amount of the isoxazole derivative contained in the above-mentioned ointment, cream, lotion, solution, suspension and gel preparation as an active ingredient is selected from the range of 0.01-10 w/w %, preferably 0.1-10 w/w %, of the entire amount of the dosage form. More preferably, it is selected from the range of 0.2-5 w/w %, more preferably from the range of 1-5 w/w %.

In addition, the ratio of the oil phase and the aqueous phase of creams is selected from the range of 1:100-100:1, preferably 10:90-80:20, in weight ratio.

The ratio of the oil phase and the aqueous phase in an emulsion lotion is selected from the range of 1:100-50:50, preferably 1:100-40:60, in a weight ratio.

When an emulsifier is used for these dosage forms, it is sufficient to use the emulsifier in the range of 0.1-20 w/w %, preferably 1-15 w/w %, of the entire amount of the dosage form.

Moreover, these ointments, creams, lotions, solutions, suspensions and gel preparations can contain, on demand, pharmaceutically acceptable various excipients, such as fragrance, filler, other transdermal penetration enhancer and the like in the range the object of the present invention is not impaired.

In the present invention, moreover, a plasters (including transdermal patch and the like) and the like can be selected as a dosage form.

The plaster is a laminate of an adhesive base on a backing layer. As the backing layer, a flexible material capable of freely following the stretch and shrinkage of the skin is preferable. For example, known ones such as plastic film, cloth, paper and the like can be mentioned. The adhesive base constituting the plaster comprises an adhesive and a tackifier and a softener added as necessary, and, a base is appropriately selected from known ones in consideration of the skin safety, adhesion to the skin and the like. For example, an adhesive can be selected from acrylic type, rubber type, silicone type and the like.

Of these, as the acrylic type, for example, a (co)polymer mainly comprising (meth)acrylic acid alkyl ester can be mentioned. This (co)polymer may be a copolymer of two or more kinds of (meth)acrylic acid alkyl esters, or a copolymer of a functional monomer capable of copolymerization with (meth)acrylate alkyl ester and (meth)acrylic acid alkyl ester.

As the rubber type, for example, those comprising a rubber adhesive as a main component, such as natural rubber, polyisopropylene rubber, polyisobutylene rubber, styrene-isoprene-styrene block copolymer, styrene-butadiene-styrene block copolymer and the like can be mentioned.

As the silicone type, for example, those comprising a silicone rubber as a main component, such as polydimethyl siloxane, diphenyl siloxane and the like can be mentioned.

As the tackifier, rosin, hydrogenated rosin, rosin ester, hydrogenated rosin ester, polyterpene resin, oil-soluble phenol resin and the like can be mentioned.

A softener plasticizes and softens the above-mentioned adhesive and tackifier to impart suitable adhesiveness to the skin. For example, almond oil, olive oil, camellia oil, persic oil, peanut oil, olefin acid, liquid paraffin and the like can be used.

A plaster can naturally contain conventionally known inorganic fillers, plasticizers, stabilizers, UV absorbers, preservatives, fragrances and the like as necessary.

In the present invention, the amount of the isoxazole derivative to be contained in the above-mentioned plaster as an active ingredient is selected from the range of 0.01-10 w/w %, preferably 0.1-10 w/w %, more preferably 0.2-5 w/w %, and still more preferably 1-5 w/w %, of the entire amount of the adhesive base.

Preferable examples of these dosage forms include ointments, creams, solutions, plasters and the like.

Furthermore, a pharmaceutical composition for transdermal administration of the present invention, which comprises leflunomide or leflunomide metabolite, or a pharmaceutically acceptable salt thereof (hereinafter sometimes to be abbreviated as leflunomides including the both in the present specification) as an active ingredient can retain the active ingredient stably by containing leflunomides in a carrier containing a particular base. As a result, the composition of the present invention characteristically shows enhanced absorbability and markedly improved object anti-inflammatory effect or antirheumatic effect.

The amount of leflunomides contained in the pharmaceutical composition for transdermal administration as an active ingredient is selected from the range of 0.01-10 w/w %, preferably 0.1-10 w/w %, of the entire amount of the dosage form. More preferably, it is selected from the range of 0.2-5 w/w %, more preferably from the range of 1-5 w/w %.

Leflunomides, which is an active ingredient of the pharmaceutical composition for transdermal administration of the present invention, shows markedly different physicochemical properties such as solubility, stability and the like depending on the base to be selected. If the active ingredient is not retained physicochemically stably in a carrier containing the selected base component, not only the preparation stability but also the absorbability and efficacy of the active ingredient may be affected.

In the present invention, by the “carrier for transdermal administration” is meant the component of the pharmaceutical composition for transdermal administration of the present invention excluding the active ingredient leflunomides.

The carrier for transdermal administration of the present invention includes “a base for dissolution” and “a hydrophobic base for suspension having no polar group in a molecule”.

When a base for dissolution is used for the pharmaceutical composition for transdermal administration of the present invention, a “soluble-type pharmaceutical composition for transdermal administration”, wherein the active ingredient is dissolved, is obtained and when a hydrophobic base for suspension having no polar group in a molecule is used, a “suspended-type pharmaceutical composition for transdermal administration” wherein the active ingredient is suspended is obtained. Each is explained in the following.

1. Soluble-Type Pharmaceutical Composition for Transdermal Administration

The “base for dissolution” to be used as one component of the carrier for transdermal administration in the “soluble-type pharmaceutical composition for transdermal administration” of the present invention is a base for retaining the active ingredient leflunomides in a preparation in a dissolution state to allow transdermal absorption of leflunomides. As the base for dissolution, for example, one kind of base or a mixture of two or more kinds thereof selected from dibasic acid dialkyl esters, polyoxyethylene polyoxypropylene glycols, medium chain fatty acid triglycerides and macrogols can be mentioned.

The “dibasic acid dialkyl esters” can be selected from those pharmaceutically acceptable. To be specific, for example, diethyl sebacate, diisopropyl sebacate, diisopropyl adipate, diisobutyl adipate and the like can be mentioned. Preferred are, for example, diethyl sebacate, diisopropyl adipate and the like.

These are generally used as an excipient for pharmaceutical preparation and commercially available from various manufacturers.

The “polyoxyethylene polyoxypropylene glycols” can be appropriately selected from those pharmaceutically acceptable. To be specific, for example, polyoxyethylene(20)polyoxypropylene(20)glycol, polyoxyethylene(3)polyoxypropylene(17)glycol, polyoxyethylene(42)polyoxypropylene(67)glycol, polyoxyethylene(54)polyoxypropylene(39)glycol, polyoxyethylene(120)polyoxypropylene(40)glycol, polyoxyethylene(160)polyoxypropylene(30)glycol, polyoxyethylene(196)polyoxypropylene(67)glycol and the like can be mentioned. Preferred are those that become liquid at 30° C., and specific examples include polyoxyethylene(20)polyoxypropylene(20)glycol and polyoxyethylene(3)polyoxypropylene(17)glycol. Particularly preferred is polyoxyethylene(20)polyoxypropylene(20)glycol can be mentioned.

These are generally used as an excipient for pharmaceutical preparation and commercially available from various manufacturers.

The “medium chain fatty acid triglycerides” can be appropriately selected from those pharmaceutically acceptable. As the “medium chain fatty acid triglycerides”, for example, triester of glycerin comprising a fatty acid having 8 to 12 carbon atoms and the like can be mentioned. Specifically, for example, fatty acid triglycerides having a single chain length such as glyceryl tricaprylate, glyceryl triisooctanoate, glyceryl trinonanoate, glyceryl tricaprate, glyceryl triundecanoate, glyceryl trilaurate and the like, mixed fatty acid esters such as glyceryl tricaprylate-caprate, glyceryl tricaprylate-caprate-laurate and the like can be mentioned. Preferred are mixed fatty acid triglycerides comprising a fatty acid having 8 to 12 carbon atoms and the like. More preferred are mixed fatty acid triglycerides comprising fatty acid having 8 to 12 carbon atoms and the like from the aspects of melting point and solubility.

These are generally used as an excipient for pharmaceutical preparation and commercially available from various manufacturers.

The “macrogols(polyethylene glycols)” can be appropriately selected from those pharmaceutically acceptable. To be specific, for example, macrogol 200, macrogol 300, macrogol 400, macrogol 1000, macrogol 1500, macrogol 1540, macrogol 4000, macrogol 6000 and the like can be mentioned. Preferred are those that become liquid at 30° C. Specifically, for example, macrogol 200, 300, or 400 and the like can be mentioned.

These are generally used as an excipient for pharmaceutical preparation and commercially available from various manufacturers.

Each of the above-mentioned bases for dissolution can be used alone or as a mixture. When plural of them are selected, the first base for dissolution is preferably dibasic acid dialkyl esters, or polyoxyethylene polyoxypropylene glycols. More preferred is dibasic acid dialkyl ester, and particularly preferred is diethyl sebacate or diisopropyl adipate.

The content of the base for dissolution in the carrier for transdermal administration in the soluble-type pharmaceutical composition for transdermal administration of the present invention is at least 40 w/w %, preferably not less than 50 w/w %, more preferably not less than 60 w/w %, of the carrier.

When the content of the base for dissolution is less than 40 w/w %, stability of solubility of the active ingredient leflunomides in the carrier for transdermal administration is degraded, and as a result, the object efficacy may not be achieved.

In the present invention, the carrier contains a base for dissolution in a proportion of at least 40 w/w %. As a result, the solution state of the active ingredient is sufficiently retained, the stability of the soluble-type pharmaceutical composition for transdermal administration becomes superior, and the transdermal absorption of the active ingredient is enhanced, as a result of which superior efficacy can be expressed.

In contrast, leflunomides, which are the active ingredient of the pharmaceutical composition for transdermal administration of the present invention, is hardly soluble in hydrocarbon oil. When hydrocarbon oil is contained at a level more than acceptable in a carrier for transdermal administration, the solubility of leflunomides rapidly decreases. As a result, in the case of a soluble-type pharmaceutical composition for transdermal administration of the present invention, leflunomides may be precipitated during preservation or crystals may grow. When the content of leflunomides is high, phase separation from the base for dissolution may occur due to the solubility. Therefore, a hydrocarbon oil in a carrier for transdermal administration at above a certain level as one component of the carrier is not preferable in view of the preparation stability and efficacy.

Specifically, when hydrocarbon oil is used as one of the base components to be used for the carrier for transdermal administration, the content thereof is preferably not more than 40 w/w % so as not to exceed the content of the base for dissolution.

As the hydrocarbon oil, a non-polar oil consisting of a carbon atom and a hydrogen atom can be mentioned. Specifically, for example, petroleum hydrocarbons such as liquid paraffin, n-paraffin, iso-paraffin, hydrocarbon gel, polybutenes, petrolatum, white petrolatum, solid paraffin, microcrystalline wax and the like, plant and animal hydrocarbon oils such as squalane, squalene and the like can be mentioned.

In the soluble-type pharmaceutical composition for transdermal administration of the present invention, a transdermal penetration enhancer can be used as a component of the carrier for transdermal administration, in addition to the above-mentioned component of the base for dissolution, within the range where the dissolution state of the active ingredient is not impaired.

As the transdermal penetration enhancer, fatty acids such as lauric acid, oleic acid, isostearic acid and the like, higher alcohols such as lauryl alcohol, oleyl alcohol, isostearyl alcohol and the like, lipophilic nonionic surfactant, or 1-menthol, 1-limonene, d-limonene, dl-limonene or other essential oils, and other known penetration enhancers and the like can be mentioned, which can be used according to the object.

In the soluble-type pharmaceutical composition for transdermal administration of the present invention, particularly, absorbability at the administration site can be improved and efficacy can be further enhanced without impairing the solubility of leflunomide, which is the active ingredient by adding a particular lipophilic nonionic surfactant.

As the lipophilic nonionic surfactant, for example, α-monoalkyl glyceryl ethers, glyceryl fatty acid monoesters, polyglyceryl fatty acid esters, sorbitan fatty acid esters and the like can be mentioned.

As the α-monoalkyl glyceryl ethers, specifically, for example, α-monolauryl glyceryl ether, α-monotridecyl glyceryl ether, α-monomyristyl glyceryl ether, α-monopentadecyl glyceryl ether, α-monocetyl glyceryl ether, α-monostearyl glyceryl ether, α-monoisostearyl glyceryl ether, α-monooleyl glyceryl ether and the like can be mentioned. Preferred is one that has a relatively low melting point and becomes liquid on the skin. Specifically, for example, α-monoisostearyl glyceryl ether, α-monooleyl glyceryl ether and the like can be mentioned.

As the glyceryl fatty acid monoesters, specifically, for example, glyceryl monolaurate, glyceryl monotridecanoate, glyceryl monomyristate, glyceryl monopentadecanoate, glyceryl monopalmitate, glyceryl monostearate, glyceryl monoisostearate, glyceryl monooleate and the like can be mentioned. Of these, preferred is one that has a relatively low melting point and becomes liquid on the skin. Specifically, for example, glyceryl monoisostearate, glyceryl monooleate and the like can be mentioned.

As the ployglyceryl fatty acid esters, specifically, for example, an ester of lauric acid, tridecanoic acid, myristic acid, pentadecanoic acid, palmitic acid, stearic acid, isostearic acid, oleic acid and the like and polyglycerol can be mentioned, like the above-mentioned glyceryl fatty acid esters. As the polyglycerol, diglycerol, triglycerol, tetraglycerol, hexaglycerol, decaglycerol and the like can be mentioned.

As the sorbitan fatty acid esters, specifically, for example, sorbitan monolaurate, sorbitan dilaurate, sorbitan monomyristate, sorbitan dimyristate, sorbitan monopalmitate, sorbitan dipalmitate, sorbitan monostearate, sorbitan distearate, sorbitan monoisostearate, sorbitan diisostearate, sorbitan monooleate, sorbitan dioleate and the like can be mentioned.

In the case of polyglyceryl fatty acid esters and sorbitan fatty acid esters, preferred is one that has a relatively low melting point and becomes liquid on the skin. Specifically, for example, isostearate and oleate can be mentioned.

As a preferable lipophilic nonionic surfactant, α-monoalkyl glyceryl ethers can be mentioned. Of these, more preferred is α-monoisostearyl glyceryl ether.

In the soluble-type pharmaceutical composition for transdermal administration of the present invention, the content of the lipophilic nonionic surfactant in a carrier for transdermal administration is selected from the range of 0.1-10 w/w %, preferably from the range of 0.2-5 w/w %, more preferably from the range of 0.5-3 w/w %.

When the content is less than 0.1 w/w %, the effect of lipophilic nonionic surfactant may not be fully achieved and when it exceeds 10 w/w %, the stability of the obtained pharmaceutical composition for transdermal administration may be degraded depending on the base to be the main component.

The amount of leflunomides contained in the soluble-type pharmaceutical composition for transdermal administration as an active ingredient is selected from the range of 0.01-10 w/w %, preferably 0.1-10 w/w %, of the entire amount of the dosage form. More preferably, it is selected from the range of 0.2-5 w/w %, more preferably from the range of 1-5 w/w %.

The final dosage form of the soluble-type pharmaceutical composition for transdermal administration of the present invention, which contains leflunomides as the active ingredient, is, for example, ointment, solution, gel preparation, plaster and the like from conventional dosage forms for external applications.

Such dosage form for external application can be manufactured by a conventional method using general base, thickener and the like. These can be manufactured by, for example, following the description of “Development manual of transdermally applicable preparations” supervised by Mitsuo Matsumoto (1985), JP-B-2651616, WO96/12465 or JP-A-9-278651.

As the ointment, for example, oleaginous ointment, hydrophilic ointment and the like can be mentioned. Components to be used for the carrier for transdermal administration are determined for each agent.

In the case of an oleaginous ointment, the base for dissolution is selected from, for example, dibasic acid dialkyl esters, medium chain fatty acid triglycerides and the like and mixtures thereof.

Specifically, it is selected from, for example, diethyl sebacate, diisopropyl adipate, which is a dibasic acid dialkyl ester, and glyceryl tricaprylate-caprate, which is a medium chain fatty acid triglyceride.

As a component of the carrier for transdermal administration, for example, fatty oils such as vegetable oil, animal oil and the like, fatty acid esters, aliphatic alcohols, polyalkylene glycols, and those generally known as a base for ointment such as hydrocarbon oil and the like can be added after appropriate selection within the range that do not inhibit the solubility and stability of leflunomides, which is the active ingredient. Specifically, for example, butyl stearate, hexadecyl isostearate, isopropyl palmitate, isostearyl palmitate, isopropyl myristate, octyldodecyl myristate, cetyl myristate, oleyl oleate, decyl oleate, glyceryl trimyristate, castor oil, hydrogenated oil, glyceryl monostearate, stearic acid, isostearic acid, stearyl alcohol, cetyl alcohol, oleyl alcohol, liquid lanolin, lanolin, purified lanolin, yellow beeswax, white beeswax, liquid paraffin, hydrocarbon gel, squalane, squalene, polybutenes, petrolatum, white petrolatum, solid paraffin, microcrystalline wax, silicon oil, hydrogenated oil and the like, and one of these bases or a mixture thereof depending on the object can be mentioned.

The oleaginous ointment can be prepared by increasing the viscosity of the carrier for transdermal administration, which is a base for dissolution, or a mixture of a base for dissolution and the above-mentioned appropriate component added as necessary.

In the case of a hydrophilic ointment, the base for dissolution is selected from, for example, polyoxyethylene polyoxypropylene glycols, macrogols (polyethylene glycols) and the like and a mixture thereof. Specifically, for example, polyoxyethylene(20)polyoxypropylene(20)glycol, polyoxyethylene(3)polyoxypropylene(17)glycol, polyoxyethylene(42)polyoxypropylene(67)glycol, polyoxyethylene(54)polyoxypropylene(39)glycol, polyoxyethylene(120)polyoxypropylene(40)glycol, polyoxyethylene(160)polyoxypropylene(30)glycol, polyoxyethylene(196)polyoxypropylene(67)glycol and the like, macrogols such as macrogol 200, macrogol 300, macrogol 400, macrogol 1500, macrogol 1540, macrogol 4000, macrogol 20000 and the like, and the like and mixtures thereof are selected.

As other components to be used as a carrier for transdermal administration, alcohols such as propylene glycol and the like, water-soluble polymers such as povidone, polyvinyl alcohol and the like, and the like, dibasic acid dialkyl esters such as diethyl sebacate, diisopropyl adipate and the like, and the like, bases for dissolution other than the above-mentioned base such as medium chain fatty acid triglyceride and the like, and the like or a mixture thereof can be added within the range that does not inhibit the solubility and stability of the active ingredient leflunomides.

As the solution, an oleaginous soluble-type solution and a hydrophilic soluble-type solution can be mentioned.

In the case of an oleaginous soluble-type solution, the base for dissolution is selected from, for example, dibasic acid dialkyl esters such as diethyl sebacate, diisopropyl adipate and the like, medium chain fatty acid triglycerides such as glyceryl tricaprylate-caprate and the like, and a mixture thereof.

As the carrier for transdermal administration to be used for the oleaginous soluble-type solution, dibasic acid dialkyl esters such as diethyl sebacate, diisopropyl adipate and the like, medium chain fatty acid triglycerides such as glyceryl tricaprylate-caprate and the like, and the like may be used in combination with polyoxyethylene polyoxypropylene glycols and the like, whereby a carrier consisting only of such base for dissolution can be prepared.

Furthermore, as other components to be used for a carrier for transdermal administration, a liquid from fatty oils such as vegetable oil, animal oil and the like, fatty acid esters, aliphatic alcohols, polyalkylene glycols, and the like, and a mixture thereof can be added within the range that does not inhibit the solubility and stability of the active ingredient leflunomides. Specifically, for example, isopropyl myristate, octyldodecyl myristate, oleyl oleate, decyl oleate, isostearic acid, oleyl alcohol, liquid lanolin, liquid paraffin, squalane, squalene, polybutenes, silicon oil and the like and a mixture thereof can be mentioned.

In the case of a hydrophilic soluble-type solution, the base for dissolution is selected from, for example, polyoxyethylene polyoxypropylene glycols, macrogols and the like and a mixture thereof.

It is also possible to add, as a component of a carrier for transdermal administration, besides the base for dissolution, for example, alcohols such as propylene glycol and the like, and the like and a mixture thereof, within the range that does not inhibit the solubility and stability of the active ingredient leflunomides.

In addition, dibasic acid dialkyl esters such as diethyl sebacate, diisopropyl adipate and the like, medium chain fatty acid triglycerides such as glyceryl tricaprylate-caprate and the like, which are bases for dissolution other than those mentioned above, and the like can be also added within the range that does not impair the homogeneity of the carrier for transdermal administration.

As the gel preparation, oleaginous gel and aqueous gel can be mentioned.

In the case of an oleaginous gel, the base for dissolution is selected from, for example, dibasic acid dialkyl esters such as diethyl sebacate, diisopropyl adipate and the like, medium chain fatty acid triglycerides such as glyceryl tricaprylate-caprate and the like, and the like and a mixture thereof.

As the components of a carrier for transdermal administration other than the base for dissolution, for example, liquids from fatty oils such as vegetable oil, animal oil and the like, fatty acid esters, aliphatic alcohols, polyalkylene glycols and the like can be also used in an appropriate combination as necessary with the aforementioned base for dissolution, within the range that does not inhibit the solubility and stability of the active ingredient leflunomides.

The oleaginous gel preparation is prepared by adding, as other component of a carrier for transdermal administration, a component for gelatinization thereof or increasing viscosity thereof to such bases for dissolution or a base mixture containing a base for dissolution. As a component for gelatinization or increasing viscosity thereof, for example, polymer compounds, metal soaps such as magnesium stearate and the like, inorganic salts, purified organic clay such as hectorites and the like, and the like and a mixture thereof can be used.

In the case of an aqueous gel, the base for dissolution is selected from, for example, polyoxyethylene polyoxypropylene glycols, macrogols and the like and a mixture thereof.

As a component of a carrier for transdermal administration other than a base for dissolution, for example, alcohols such as propylene glycol and the like, and the like can be also used in an appropriate combination as necessary with the aforementioned base for dissolution, within the range that does not inhibit the solubility and stability of the active ingredient leflunomides.

Aqueous gel preparation is prepared by adding, as a component of a carrier for transdermal administration, a component for gelatinization thereof or increasing viscosity thereof to such bases for dissolution or a base mixture containing a base for dissolution. As a component for gelatinization thereof or increasing viscosity thereof, for example, aqueous polymers such as carboxymethyl polymer, hydroxypropyl cellulose, polyvinyl alcohol and the like can be added.

For the soluble-type pharmaceutical composition for transdermal administration of the present invention, a dosage form such as plasters and the like can be further employed.

The plaster is a laminate of an adhesive base on a backing layer. As the backing layer, a flexible material capable of freely following the stretch and shrinkage of the skin is preferable. For example, known ones such as plastic film (polyethylene, polyethylene terephthalate, polypropylene and the like), nonwoven cloth, cloth, paper and the like can be mentioned.

The adhesive base constituting the plaster comprises a base for dissolution and an adhesive, or a tackifier and a softener added as necessary, and, a base is appropriately selected from known ones in consideration of the skin safety, adhesion to the skin and the like.

As the base for dissolution to be used for the plaster, for example, dibasic acid dialkyl esters such as diethyl sebacate, diisopropyl adipate and the like, medium chain fatty acid triglycerides such as glyceryl tricaprylate-caprate and the like, and the like and a mixture thereof can be employed.

For example, an adhesive can be selected from acrylic type, rubber type, silicone type and the like.

Of these, as the acrylic type, for example, a (co)polymer mainly comprising (meth)acrylic acid alkyl ester can be mentioned. This (co)polymer may be a copolymer of two or more kinds of (meth)acrylic acid alkyl esters, or a copolymer of a functional monomer capable of copolymerization with (meth)acrylate alkyl ester and (meth)acrylic acid alkyl ester.

As the rubber type, for example, those comprising a rubber adhesive as a main component, such as natural rubber, polyisopropylene rubber, polyisobutylene rubber, styrene-isoprene-styrene block copolymer, styrene-butadiene-styrene block copolymer and the like can be mentioned.

As the silicone type, for example, those comprising a silicone rubber as a main component, such as polydimethyl siloxane, diphenyl siloxane and the like can be mentioned.

As the tackifier, rosin, hydrogenated rosin, rosin ester, hydrogenated rosin ester, polyterpene resin, oil-soluble phenol resin and the like can be mentioned.

A softener plasticizers and softens the above-mentioned adhesive and tackifier to impart suitable adhesiveness to the skin. For example, almond oil, olive oil, camellia oil, persic oil, peanut oil, olefin acid, liquid paraffin and the like can be used.

The thickness of the base to be laminated on the backing layer of a plaster is selected from the range of 1-1000 μm, preferably from the range of 10-500 μm, and more preferably from the range of 20-200 μm.

The amount of leflunomides contained in the ointments, solutions, gel preparations, plasters as an active ingredient is selected from the range of 0.01-10 w/w %, preferably 0.1-10 w/w %, of the entire amount of the dosage form. More preferably, it is selected from the range of 0.2-5 w/w %, more preferably from the range of 1-5 w/w %.

These ointments, solutions, gel preparations and plasters can contain, as a component of a carrier for transdermal administration, pharmaceutically acceptable various excipients, such as fragrance, filler, humectant, stabilizer, preservative, emulsifier and the like on demand, within the range that does not impair the object of the present invention.

As the humectant, for example, propylene glycol, glycerin, 1,3-butylene glycol and the like can be mentioned.

As the stabilizer, for example, dibutylhydroxytoluene, dl-α-tocopherol, tocopherol acetate, propyl gallate and the like can be mentioned.

As the preservative, for example, methylparaben, ethylparaben, propylparaben, chlorobutanol, benzyl alcohol and the like can be mentioned.

As the emulsifier, one appropriately selected from those generally used for pharmaceutical products can be used. Specifically, for example, sucrose fatty acid esters; polysorbates such as polyoxyethylene sorbitan monostearate, polyoxyethylene sorbitan monooleate, polyoxyethylene sorbitan trioleate and the like; polyoxyethylene alkyl ethers such as polyoxyethylene cetyl ether, polyoxyethylene oleyl ether and the like; polyoxyethylene fatty acid esters such as polyoxyl 40 stearate, polyethyleneglycol monolaurate and the like; and the like can be mentioned.

The soluble-type pharmaceutical composition for transdermal administration of the present invention is prepared by dissolving the active ingredient leflunomides in a particular base for dissolution. Specifically, for example, leflunomides is added to a base for dissolution and sufficiently dissolved therein. Depending on the dosage form employed, various bases, excipients and the like may be added to give a dosage form of ointment, solution, gel preparation and the like. The preparation conditions such as temperature, stirring force and the like can be selected from the conditions suitable for each dosage form. Ointments are generally prepared in the temperature range of from near room temperature to about 80° C., solutions are generally prepared in the temperature range of from near room temperature to about 50° C., and gel preparations are generally prepared in the temperature range of from near room temperature to about 60° C. In addition, plasters are generally prepared by, for example, adding leflunomides to a base for dissolution to allow sufficient dissolution therein, mixing the solution with adhesive, or tackifier and softener as necessary, as mentioned above, to give an adhesive base and laminating the base on a backing layer such as a plastic film and the like. As the preparation method of plasters, hot melt methods and solvent methods can be mentioned, which may be appropriately employed for preparation depending on the object. In general, the hot melt method employs the range of about 150° C. to about 250° C. depending on the kind of elastomer to be used and the solvent method employs the range of up to about 60° C. for, for example, hexane.

Examples of the dosage form of the soluble-type pharmaceutical composition for transdermal administration of the present invention include ointments, solutions, gel preparations, plasters and the like, with preference given to ointments, solutions and plasters, which are particularly preferably solutions and plasters.

The soluble-type pharmaceutical composition for transdermal administration of the present invention is capable of stably dissolving the active ingredient leflunomides by employing these dosage forms and as a result, can provide superior absorbability and efficacy.

The soluble-type pharmaceutical composition for transdermal administration of the present invention is advantageous in that the content homogeneity of the active ingredient is easily ensured and the production is easy.

2. Suspended-Type Pharmaceutical Composition for Transdermal Administration

The suspended-type pharmaceutical composition for transdermal administration of the present invention contains the active ingredient leflunomides in a suspension state.

In the suspended-type pharmaceutical composition for transdermal administration of the present invention, the carrier for transdermal administration is one or more kinds selected from hydrophobic base for suspension having no polar group in a molecule, or a mixture of these bases for suspension and other components necessary for forming a preparation.

Particularly, leflunomides have weak affinity for a hydrophilic or hydrophobic substance having various polar groups. Therefore, when a substance having a polar group is contained at a level not less than the acceptable level in the “carrier for transdermal administration” to be suspended, the solubility of leflunomides increases, and as a result, unpreferable changes in the form such as crystal growth, phase separation and the like occur during preservation.

The “polar group” in the present specification specifically refers to, for example, hydroxyl group, carboxyl group, carbonyl group, amino group, amine group, ammonium group, halogen group, acidic group (e.g., sulfonyl group, phosphoryl group etc.) and various metal salts.

The hydrophobic base for suspension having no polar group in a molecule and which is used for the “carrier for transdermal administration” can be selected from pharmaceutically acceptable various bases. In consideration of dissolution property and stability of leflunomides, hydrocarbon oil is preferably selected, and one having a high viscosity is more preferable.

As the “hydrocarbon oil”, for example, petrolatum, white petrolatum, purified petrolatum, n-paraffin, isoparaffin, liquid paraffin, hydrocarbon gel, microcrystalline wax, squalane, squalene, polybutenes, polyisoprene and the like can be mentioned, with preference given to hydrocarbon gel.

The “hydrocarbon gel” is obtained by gelatinization of heavy liquid paraffin, which is a liquid hydrocarbon oil, with polyethylene, and is commercially available as PLASTIBASE (trademark).

The content of a “hydrophobic base for suspension having no polar group in a molecule” to be used for the “carrier for transdermal administration” in the suspended-type pharmaceutical composition for transdermal administration of the present invention is selected from the range of not less than 70 w/w %, preferably not less than 80 w/w %, more preferably not less than 90 w/w %, particularly preferably not less than 95 w/w %, of the carrier.

In the present invention, a carrier containing a “hydrophobic base for suspension having no polar group in a molecule” as a main component is selected to prepare a suspended-type pharmaceutical composition for transdermal administration, which is particularly superior in the stability of leflunomides.

The shape of the leflunomides to be used as the active ingredient in the suspended-type pharmaceutical composition for transdermal administration of the present invention is not particularly limited. When a suspended-type preparation is prepared, pulverization of initial particle of a dispersed substance is not accompanied, and therefore, leflunomides to be suspended in the carrier for transdermal administration of the present invention preferably have a particle size of not more than 100 μm. Preferably, one wherein all the particles substantively have a particle size of not more than 100 μm, and an average particle size thereof is not more than 20 μm, is selected. More preferably, one wherein all the particles have a particle size of substantively not more than 20 μm, and an average particle size thereof is not more than 10 μm is selected.

When the particle size of the active ingredient exceeds 100 μm, the uniformity and transdermal absorbability of the pharmaceutical composition finally obtained may be impaired.

By the “substantively all the particles have a particle size of not more than 100 μm, or not more than 20 μm” is meant, for example, particle size distribution wherein not less than 90% of the entire particle has a particle size of not more than 100 μm or not more than 20 μm, by measurement of the particle size based on the volume. In addition, the average particle size refers to an average volume particle size.

In the present specification, the average particle size and particle size distribution are measured with a laser diffraction particle size analyzer and the specific measurement method thereof is as described in Examples.

To afford a desired particle size, a bulk of the active ingredient is pulverized with a jet mill and the like.

In the suspended-type pharmaceutical composition for transdermal administration of the present invention, leflunomides as an active ingredient is stably suspended in a carrier for transdermal administration. The content thereof can be selected from a wide range, without consideration of solubility and the like of leflunomides.

The amount of leflunomides to be contained as an active ingredient in the suspended-type pharmaceutical composition for transdermal administration of the present invention is specifically selected from the range of 0.01-20 w/w % of the entire amount of the composition, and preferably selected from the range of 0.1-10 w/w %, more preferably from the range of 0.5-10 w/w %, and still more preferably from the range of 1-5 w/w %.

The dosage form of the suspended-type pharmaceutical composition for transdermal administration of the present invention is a conventional dosage form for external application, which is determined according to the viscosity of the “hydrophobic base for suspension having no polar group in a molecule”. Examples thereof include ointments, liquids, plasters and the like.

The dosage form for external application can be produced using conventional bases, thickeners and the like according to conventional methods. They can be produced according to, for example, “Transdermally Applicable Preparation Development Manual” ed. Mitsuo Matsumoto (1985), JP-B-2651616, WO96/12465, JP-A-9-278651 and the like.

As the ointment, for example, a suspended oleaginous ointment can be mentioned.

As the “hydrophobic base for suspension having no polar group in a molecule” to be used for a suspended oleaginous ointment, for example, bases for suspension without a polar group such as petrolatum, white petrolatum, purified petrolatum, paraffin, liquid paraffin, hydrocarbon, hydrocarbon gel, squalene, squalane, polybutenes and the like can be mentioned.

As the component other than those, for example, those generally known as a base for ointment, such as fatty oils (e.g., vegetable oil, animal oil and the like), fatty acid esters, aliphatic alcohols, polyalkylene glycols and the like can be used within the range that does not inhibit the suspensibility and stability of the active ingredient leflunomides.

As the component other than the “hydrophobic base for suspension having no polar group in a molecule”, which can be used for the suspended-type pharmaceutical composition for transdermal administration of the present invention, for example, lanolin, liquid lanolin, purified lanolin, yellow beeswax, white beeswax, diethyl sebacate, diisopropyl adipate, medium chain fatty acid triglycerides (e.g., glyceryl tricaprylate-caprate, butyl stearate, hexadecyl isostearate, isopropyl palmitate, isostearyl palmitate, isopropyl myristate, octyldodecyl myristate, cetyl myristate, oleyl oleate, decyl oleate, glyceryl trimyristate), castor oil, hydrogenated oil, glyceryl monostearate, stearic acid, isostearic acid, stearyl alcohol, cetyl alcohol, oleyl alcohol, silicon oil and the like can be mentioned. One or more of these can be appropriately used according to the object.

The content of the above-mentioned component other than the “hydrophobic base for suspension having no polar group” preferably does not exceed 30 w/w % relative to the carrier.

As the suspended liquid, for example, a suspended oleaginous liquid can be mentioned, and a base to be used for the carrier is selected according to the object, as in the above-mentioned ointment.

The “hydrophobic base for suspension having no polar group in a molecule” to be used for a suspended oleaginous liquid is a liquid base for suspension without a polar group selected from, for example, liquid paraffin, hydrocarbon, squalene, squalane, polybutenes and the like.

As a component other than those, a liquid from those generally known as bases for ointment, such as fatty oils (e.g., vegetable oil, animal oil and the like), fatty acid esters, aliphatic alcohols, polyalkylene glycols and the like can be used within the range that does not inhibit the suspensibility and stability of the active ingredient leflunomides.

As a component other than the “hydrophobic base for suspension having no polar group in a molecule” that can be used for a suspended oleaginous liquid, for example, medium chain fatty acid triglycerides such as diethyl sebacate, diisopropyl adipate, glyceryl tricaprylate-caprate and the like, isopropyl myristate, octyldodecyl myristate, oleyl oleate, decyl oleate, glyceryl trimyristate, castor oil, isostearic acid, oleyl alcohol, silicon oil and the like can be specifically mentioned. One or more of these can be appropriately used according to the object.

The content of the above-mentioned component other than the “hydrophobic base for suspension having no polar group in a molecule” preferably does not exceed 30 w/w % of the carrier.

The suspended-type plaster is a laminate comprising an adhesive base using a “hydrophobic base for suspension having no polar group in a molecule” formed on a backing layer. As the backing layer, a flexible material capable of freely following the stretch and shrinkage of the skin is preferable. For example, known ones such as plastic film (polyethylene, polyethylene terephthalate, polypropylene and the like), nonwoven cloth, cloth, paper and the like can be mentioned.

The adhesive base constituting the plaster comprises a base for suspension and an adhesive, or a tackifier and a softener added as necessary, and, a base is appropriately selected from known ones in consideration of the skin safety, adhesion to the skin and the like.

As the “hydrophobic base for suspension having no polar group in a molecule” to be used for a plaster, for example, petrolatum, white petrolatum, purified petrolatum, paraffin, liquid paraffin, hydrocarbon, hydrocarbon gel, squalene, squalane, polybutenes and the like can be used.

For example, an adhesive can be selected from acrylic type, rubber type, silicone type and the like.

Of these, as the acrylic type, for example, a (co)polymer mainly comprising (meth)acrylic acid alkyl ester can be mentioned. This (co)polymer may be a copolymer of two or more kinds of (meth)acrylic acid alkyl esters, or a copolymer of a functional monomer capable of copolymerization with (meth)acrylate alkyl ester and (meth)acrylic acid alkyl ester.

As the rubber type, for example, those comprising a rubber adhesive as a main component, such as natural rubber, polyisopropylene rubber, polyisobutylene rubber, styrene-isoprene-styrene block copolymer, styrene-butadiene-styrene block copolymer and the like can be mentioned.

As the silicone type, for example, those comprising a silicone rubber as a main component, such as polydimethyl siloxane, diphenyl siloxane and the like can be mentioned.

As the tackifier, rosin, hydrogenated rosin, rosin ester, hydrogenated rosin ester, polyterpene resin, oil-soluble phenol resin and the like can be mentioned.

The thickness of the adhesive base to be laminated on the backing layer of a suspended-type plaster is selected from the range of 1-1000 μm, preferably from the range of 10-500 μm, and more preferably from the range of 20-200 μm.

The suspended ointments, suspended liquids or -suspended plasters can contain a “lipophilic nonionic surfactant” and the like within the range that does not inhibit the suspensibility and stability of the active ingredient leflunomides.

As the “lipophilic nonionic surfactant”, specifically, for example, sorbitan fatty acid esters such as sorbitan monostearate, sorbitan monopalmitate, sorbitan monooleate, sorbitan sesquioleate and the like; glycerin fatty acid esters such as glyceryl monostearate, glyceryl monoisostearate, glyceryl monooleate and the like; butyl alcohol; Selachyl Alcohol; α-monoalkyl glyceryl ethers such as α-monoisostearyl glyceryl ether and the like; sucrose fatty acid esters; polysorbates such as polyoxyethylene sorbitan monostearate, polyoxyethylene sorbitan monooleate, polyoxyethylene sorbitan trioleate and the like; polyoxyethylene alkyl ethers such as polyoxyethylene cetyl ether, polyoxyethylene oleyl ether and the like, polyoxyethylene fatty acid esters such as polyoxyl stearate, polyethyleneglycol monolaurate and the like and the like can be mentioned.

The content of the above-mentioned “lipophilic nonionic surfactant” combined with the content of the above-mentioned component other than the “hydrophobic base for suspension having no polar group in a molecule” is desirably set to not exceed 30 w/w % of the carrier.

In the suspended-type pharmaceutical composition for transdermal administration of the present invention, addition of these lipophilic nonionic surfactants has an effect of improving the affinity for the skin.

Of the dosage forms of the suspended-type pharmaceutical composition for transdermal administration, particularly preferred is ointment.

The suspended-type pharmaceutical composition for transdermal administration of the present invention, which contains leflunomides, when prepared as an ointment having a suitable viscosity, maintain dispersion stability of leflunomides suspended as an active ingredient in a carrier, and can retain leflunomides for a long term on the skin even after administration, and as a result, can express superior efficacy.

The amount of leflunomides contained in the above-mentioned ointment, liquid or plaster as an active ingredient is selected from the range of 0.01-10 w/w %, preferably 0.1-10 w/w %, of the entire amount of the dosage form. More preferably, it is selected from the range of 0.2-5 w/w %, more preferably from the range of 1-5 w/w %.

The suspended-type pharmaceutical composition for transdermal administration of the present invention is prepared by suspending or dispersing the active ingredient leflunomides in a particular base for suspension. Specifically, for example, a sufficiently pulverized bulk of leflunomides is added to a base for suspension and stirred sufficiently. Thereafter, various bases, additives and the like are added according to the determined dosage form to give a dosage form of ointment, liquid and the like. Preparation conditions of temperature, stirring force and the like can be selected from the conditions suitable for each dosage form. However, since excessive heating may change the crystal state of suspended leflunomides as an active ingredient in a carrier, it is preferably prepared in the range of, for example, near room temperature to about 50° C.

An ointment can be also prepared using, for example, hybrid de-foaming mixer (Awatorineritaro, trademark, AR-250, THINKY corporation) near room temperature. In addition, it can be also prepared by sufficiently stirring using a vacuum emulsifier and the like while controlling the temperature.

A suspended plaster can be prepared by, for example, adding a sufficiently pulverized bulk of leflunomides to a base for suspension and sufficiently stirring therein to allow suspension and dispersion, mixing same with an adhesive, or with a tackifier and a softener as necessary to give an adhesive base and laminating the base on a backing layer such as a plastic film and the like. A plaster can be prepared by a hot melt method or a solvent method, which is appropriately determined according to the object. Preparation conditions such as temperature and the like can be selected from the conditions suitable for each preparation method. However, since excessive heating may change the crystal state of leflunomides suspended as an active ingredient in a carrier, it is preferably prepared in the range of, for example, near room temperature to about 50° C. by solvent methods.

Since dosage forms for external application, soluble-type pharmaceutical composition for transdermal administration and suspended-type pharmaceutical composition for transdermal administration and the like of the present invention have a superior anti-inflammatory effect as mentioned above, the disease to be the treatment subject is not particularly limited. However, it is particularly superior for the treatment of chronic rheumatism or arthritis.

The dosage form for external application and a pharmaceutical composition for transdermal administration of the present invention may be applied to any part of the skin as long as it is the skin, but application to the lesion or nearby skin is preferable.

The dose is appropriately determined according to the age, body weight and sex of patients, condition, dosage form and administration site.

Generally, administration of 1 mg-1.0 g/day in the amount of SMP-114 or leflunomides to an adult is performed.

### EXAMPLES

The present invention is explained in detail in the following by referring to Examples, which are not to be construed as limitative.

Leflunomide was pulverized with a jet mill and used for the preparation of the suspended-type pharmaceutical compositions for transdermal administration of Examples 22B-28B and Comparative Examples 4B, 5B. This bulk was analyzed with a laser diffraction particle size analyzer (SALD-3000, Shimadzu Corporation). As a result, the mean particle size was 7 μm and not less than 90% of the particles had a particle size of not more than 20 μm. (measurement condition: dry type, refractive index: 1.70-0.00i).

### Example 1A

SMP-114 Ointment (1A)

White petrolatum and paraffin at the following amount ratio were melted at about 80° C., cooled to about 60° C. and liquid paraffin and SMP-114 were added while maintaining the temperature and cooled with stirring until the mixture solidifies. As a result, a 5% ointment (1A) of SMP-114 was prepared.

| | |
|---|---|
| white petrolatum | 72 g |
| paraffin | 3 g |
| liquid paraffin | 20 g |
| SMP-114 | 5 g |

### Example 2A

SMP-114 Cream (1A)

White petrolatum and stearyl alcohol at the following amount ratio were melted at about 80° C., cooled to about 60° C. and components other than purified water were added. While stirring the mixture, purified water at about 50-55° C. was added gradually to the total amount of 100 g, which was cooled with stirring until it solidified. As a result, a 3% cream (1A) of SMP-114 was prepared.

| | | |
|---|---|---|
| white petrolatum | 25 | g |
| stearyl alcohol | 20 | g |
| polyoxyethylene hydrogenated castor oil 60 | 4 | g |
| glyceryl monostearate | 1 | g |
| propylene glycol | 12 | g |
| SMP-114 | 3 | g |
| purified water | total amount 100 | g |

### Example 3A

SMP-114 Lotion (1A)

Glycerine, ethanol, and purified water (about 30 mL) at the following amount ratio were sufficiently admixed. SMP-114 was added and the mixture was further stirred, and the total amount was adjusted to 100 mL with purified water. As a result, a 1% lotion (1A) of SMP-114 was prepared.

| | | |
|---|---|---|
| glycerin | 10 | g |
| ethanol | 45 | g |
| SMP-114 | 1 | g |
| purified water | total amount 100 | mL |

### Example 4A

SMP-114 Ointment (2A)

White petrolatum and paraffin at the following amount ratio were melted at about 80° C., cooled to about 60° C. while maintaining the temperature. Isopropyl myristate and SMP-114 were added, and the mixture was cooled with stirring until it solidified. As a result, a 5% ointment (2A) of SMP-114 was prepared.

| | |
|---|---|
| white petrolatum | 77 g |
| paraffin | 3 g |
| isopropyl myristate | 15 g |
| SMP-114 | 5 g |

### Example 5A

SMP-114 Ointment

White petrolatum and paraffin at the following amount ratio were melted at about 80° C., cooled to about 60° C. while maintaining the temperature. Isopropyl myristate and SMP-114 were added, and the mixture was cooled with stirring until it solidified. As a result, a 5% ointment (3A) of SMP-114 was prepared.

| | |
|---|---|
| white petrolatum | 60 g |
| paraffin | 5 g |
| isopropyl myristate | 30 g |
| SMP-114 | 5 g |

### Example 6A

SMP-114 Ointment

White petrolatum and paraffin at the following amount ratio were melted at about 80° C., cooled to about 60° C. while maintaining the temperature. Squalane and SMP-114 were added, and the mixture was cooled with stirring until it solidified. As a result, a 5% ointment (4A) of SMP-114 was prepared.

| | |
|---|---|
| white petrolatum | 77 g |
| paraffin | 3 g |
| squalane | 15 g |
| SMP-114 | 5 g |

### Example 7A

SMP-114 Suspension (1A) SMP-114 at the following amount ratio was mixed with isopropyl myristate to give a 5% suspension (1A) of SMP-114.

| | |
|---|---|
| isopropyl myristate | 1900 mg |
| SMP-114 | 100 mg |

### Example 8A

SMP-114 Suspension (2A)

SMP-114 at the following amount ratio was mixed with glycerin to give a 5% suspension (2A) of SMP-114.

| | |
|---|---|
| glycerin | 1900 mg |
| SMP-114 | 100 mg |

### Example 9A

SMP-114 Solution (1A)

SMP-114 at the following amount ratio was mixed with diisopropyl sebacate to give a 5% solution (1A) of SMP-114.

| | |
|---|---|
| diisopropyl sebacate | 1900 mg |
| SMP-114 | 100 mg |

### Example 10A

Leflunomide Suspension (1A)

Mixed with liquid paraffin at the following amount ratio to give a 5% suspension (1A) of leflunomide.

| | |
|---|---|
| liquid paraffin | 1900 mg |
| leflunomide | 100 mg |

### Example 11A

Leflunomide Ointment (1A)

White petrolatum and white beeswax at the following amount ratio were melted at about 80° C. and sorbitan sesquioleate and leflunomide were added while maintaining the temperature at about 50-60° C. The mixture was cooled with stirring until it solidified. As a result, a 5% ointment of leflunomide (1A) was prepared.

| | |
|---|---|
| white petrolatum | 88 g |
| white beeswax | 5 g |
| sorbitan Sesquioleate | 2 g |
| leflunomide | 5 g |

### Example 12A

Leflunomide Ointment (2A)

White petrolatum and paraffin at the following amount ratio were melted at about 80° C., cooled to about 60° C. while maintaining the temperature. Liquid paraffin and leflunomide were added, and the mixture was cooled with stirring until it solidified. As a result, a 1% ointment of leflunomide (2A) was prepared.

| | |
|---|---|
| white petrolatum | 76 g |
| paraffin | 3 g |
| liquid paraffin | 20 g |
| leflunomide | 1 g |

### Example 13A

Leflunomide Ointment (3A)

White petrolatum and paraffin at the following amount ratio were melted at about 80° C., cooled to about 60° C. while maintaining the temperature. Liquid paraffin and leflunomide were added, and the mixture was cooled with stirring until it solidified. As a result, a 5% ointment (3A) of leflunomide was prepared.

| | |
|---|---|
| white petrolatum | 72 g |
| paraffin | 3 g |
| liquid paraffin | 20 g |
| leflunomide | 5 g |

### Example 1B

Soluble-Type Solution of Leflunomide (1B)

Diethyl sebacate (product name: NIKKOL DES-SP, Nikko Chemicals Co., Ltd.) (99 g) was heated to about 40° C., and leflunomide (1 g) was added with stirring. After confirmation of dissolution, the mixture was cooled to room temperature to give a 1% soluble-type solution A of leflunomide.

### Example 2B

Soluble-Type Solution of Leflunomide (2B)

Diethyl sebacate (product name: NIKKOL DES-SP, Nikko Chemicals Co., Ltd.) (95 g) was heated to about 40° C., and leflunomide (5 g) was added with stirring. After confirmation of dissolution, the mixture was cooled to room temperature to give a 5% soluble-type solution B of leflunomide.

### Example 3B

Soluble-Type Solution of Leflunomide (3B)

Diethyl sebacate (product name: NIKKOL DES-SP, Nikko Chemicals Co., Ltd.) (50 g) and diisopropyl adipate (product name: NIKKOL DID, Nikko Chemicals Co., Ltd.) (45 g) were mixed at room temperature, and leflunomide (5 g) was added with stirring. After confirmation of dissolution, 5% soluble-type solution C of leflunomide was obtained.

### Example 4B

Soluble-Type Solution of Luflunomide (4B)

While stirring polyoxyethylene(20)polyoxypropylene(20)glycol (product name: ADEKA PLURONIC L-44, Asahi Denka Co., Ltd.) (95 g), leflunomide (5 g) was added at room temperature. After confirmation of dissolution, a 5% soluble-type solution D of leflunomide was obtained.

### Example 5B

Soluble-Type Solution of Luflunomide (5B)

While stirring polyoxyethylene(20)polyoxypropylene(20)glycol (product name: ADEKA PLURONIC L-44, Asahi Denka Co., Ltd.) (90 g), leflunomide (10 g) was added at room temperature. After confirmation of dissolution, a 10% soluble-type solution E of leflunomide was obtained.

### Example 6B

Soluble-Type Solution of Leflunomide (6B)

Diethyl sebacate (product name: NIKKOL DES-SP, Nikko Chemicals Co., Ltd.) (55 g) and polyoxyethylene(20)polyoxypropylene(20)glycol (product name: ADEKA PLURONIC L-44, Asahi Denka Co., Ltd.) (40 g) were mixed at room temperature, and leflunomide (5 g) was added with stirring. After confirmation of dissolution, a 5% soluble-type solution F of leflunomide was obtained.

### Example 7B

Soluble-Type Solution of Leflunomide (7B)

Diethyl sebacate (product name: NIKKOL DES-SP, Nikko Chemicals Co., Ltd.) (95 g) and α-monoisostearyl glyceryl ether (product name: PENETOL GE-IS, Kao Corporation) (2.5 g) were mixed at room temperature, and leflunomide (2.5 g) was added. After confirmation of dissolution, a 2.5% soluble-type solution G of leflunomide was obtained.

### Example 8B

Soluble-Type Solution of Leflunomide (8B)

Diethyl sebacate (product name: NIKKOL DES-SP, Nikko Chemicals Co., Ltd.) (92.5 g) and α-monoisostearyl glyceryl ether (product name: PENETOL GE-IS, Kao Corporation) (2.5 g) were mixed at room temperature, and leflunomide (5 g) was added with stirring. After confirmation of dissolution, a 5% soluble-type solution H of leflunomide was obtained.

### Example 9B

Soluble-Type Solution of Leflunomide (9B)

Polyoxyethylene(20)polyoxypropylene(20)glycol (product name: ADEKA PLURONIC L-44, Asahi Denka Co., Ltd.) (92.5 g) and α-monoisostearyl glyceryl ether (product name: PENETOL GE-IS, Kao Corporation) (2.5 g) were mixed at room temperature, and leflunomide (5 g) was added with stirring. After confirmation of dissolution, a 5% soluble-type solution I of leflunomide was obtained.

### Example 10B

Soluble-Type Solution of Leflunomide (10B)

Diethyl sebacate (product name: NIKKOL DES-SP, Nikko Chemicals Co., Ltd.) (52.5 g) and ethanol (42.5 g) were mixed at room temperature, and leflunomide (5 g) was added with stirring. After confirmation of dissolution, a 5% soluble-type solution J of leflunomide was obtained.

### Example 11B

Soluble-Type Solution of Leflunomide (11B)

Diethyl sebacate (product name: NIKKOL DES-SP, Nikko Chemicals Co., Ltd.) (40 g), diisopropyl adipate (product name: NIKKOL DID, Nikko Chemicals Co., Ltd.) (12 g), polyoxyethylene(20)polyoxypropylene(20)glycol (product name: ADEKA PLURONIC L-44, Asahi Denka Co., Ltd.) (30 g), propylene glycol (Asahi Denka Co., Ltd.) (13 g) and α-monoisostearyl glyceryl ether (product name: PENETOL GE-IS, Kao Corporation) (2.5 g) were mixed at room temperature, and leflunomide (2.5 g) was added with stirring. After confirmation of dissolution, a 2.5% soluble-type solution K of leflunomide was obtained.

### Example 12B

Soluble-Type Solution of Leflunomide (12B)

Diethyl sebacate (product name: NIKKOL DES-SP, Nikko Chemicals Co., Ltd.) (40 g), diisopropyl adipate (product name: NIKKOL DID, Nikko Chemicals Co., Ltd.) (12 g), medium chain fatty acid triglyceride (product name: ODO, The Nisshin OilliO Group, Ltd.) (30 g), isopropyl myristate (product name: NIKKOL IPM-100, Nikko Chemicals Co., Ltd.) (13 g) and α-monoisostearyl glyceryl ether (product name: PENETOL GE-IS, Kao Corporation) (2.5 g) were mixed at room temperature, and leflunomide (2.5 g) was added with stirring. After confirmation of dissolution, a 2.5% soluble-type solution L of leflunomide was obtained.

### Example 13B

Soluble-Type Solution of Leflunomide (13B)

Diisopropyl adipate (product name: NIKKOL DID, Nikko Chemicals Co., Ltd.) (12 g), polyoxyethylene(20)polyoxypropylene(20)glycol (product name: ADEKA PLURONIC L-44, Asahi Denka Co., Ltd.) (53 g), propylene glycol (Asahi Denka Co., Ltd.) (30 g) and α-monoisostearyl glyceryl ether (product name: PENETOL GE-IS, Kao Corporation) (2.5 g) were mixed at room temperature, and leflunomide (2.5 g) was added with stirring. After confirmation of dissolution, a 2.5% soluble-type solution M of leflunomide was obtained.

### Example 14B

Soluble-Type Solution of Leflunomide (14B)

Diisopropyl adipate (product name: NIKKOL DID, Nikko Chemicals Co., Ltd.) (34.5 g), polyoxyethylene(20)polyoxypropylene(20)glycol (product name: ADEKA PLURONIC L-44, Asahi Denka Co., Ltd.) (30 g), macrogol 400 (product name: macrogol 400, NOF CORPORATION) (27.5 g), α-monooleyl glyceryl ether (product name: NIKKOL Selachyl Alcohol, Nikko Chemicals Co., Ltd.) (2.5 g) were mixed with heating to 40° C., and dibutyl hydroxytoluene (product name: YOSHINOX BHT, API Corporation) (0.5 g) was added with stirring. After complete dissolution, leflunomide (5 g) was further added. After confirmation of dissolution, a 5% soluble-type solution N of leflunomide was obtained.

### Example 15B

Soluble-Type Solution of Leflunomide (15B)

Diethyl sebacate (product name: NIKKOL DES-SP, Nikko Chemicals Co., Ltd.) (65 g) and leflunomide (5 g) were mixed and stirred at room temperature to dissolve leflunomide. Liquid paraffin (YUKA SANGYO CO., LTD.) (30 g) was further added with stirring to give a 5% soluble-type solution O of leflunomide.

### Example 16B

Soluble-Type Solution of Leflunomide (16B)

Diethyl sebacate (product name: NIKKOL DES-SP, Nikko Chemicals Co., Ltd.) (40 g) and leflunomide (2.5 g) were mixed and stirred at room temperature. After confirmation of dissolution, diisopropyl adipate (product name: NIKKOL DID, Nikko Chemicals Co., Ltd.) (12 g), medium chain fatty acid triglyceride (product name: ODO, The Nisshin OilliO Group, Ltd.) (30 g), liquid paraffin (YUKA SANGYO CO. LTD.) (13 g) and α-monoisostearyl glyceryl ether (product name: PENETOL GE-IS, Kao Corporation) (2.5 g) were further added with stirring and uniformly dissolved to give a 2.5% soluble-type solution P of leflunomide.

### Example 17B

Soluble-Type Ointment of Leflunomide (1B)

Polyoxyethylene(20)polyoxypropylene(20)glycol (product name: ADEKA PLURONIC L-44, Asahi Denka Co., Ltd.) (50 g), macrogol 400 (product name: macrogol 400, NOF CORPORATION) (10 g), diisopropyl adipate (product name: NIKKOL DID, Nikko Chemicals Co., Ltd.) (10 g) and leflunomide (5 g) were mixed at room temperature, dissolved, and heated to about 60° C. With stirring, this was slowly added to a mixture of macrogol 4000 (product name: macrogol 4000, NOF CORPORATION) (14 g) and stearyl alcohol (product name: KALCOL 8688, Kao Corporation) (11 g) melted in advance by heating to about 70° C. With stirring, the mixture was gradually cooled to solidness to give a 5% soluble-type ointment Q of leflunomide.

### Example 18B

Soluble-Type Ointment of Leflunomide (2B)

Macrogol 400 (product name: macrogol 400, NOF CORPORATION) (45 g) and leflunomide (5 g) were mixed at room temperature, dissolved, and heated to about 60° C. With stirring, this was slowly added to a mixture of macrogol 4000 (product name: macrogol 4000, NOF CORPORATION) (50 g) melted in advance by heating to about 70° C., and dissolution thereof was confirmed. With stirring, the mixture was gradually cooled to solidness to give of a 5% soluble-type ointment R of leflunomide.

### Example 19B

Soluble-Type Gel Preparation of Leflunomide (1B)

Diethyl sebacate (product name: NIKKOL DES-SP, Nikko Chemicals Co., Ltd.) (80 g) was heated to about 40° C. and leflunomide (5 g) was added with stirring. After confirmation of dissolution, dimethyl distearylammonium hectorite (product name: LUCENTITE SAN, CO-OP CHEMICALS CO., LTD) (15 g) was added, and cooled with sufficient stirring to give a 5% soluble-type gel preparation S of leflunomide.

### Example 20B

Soluble-Type Plaster of Leflunomide (1B)

Diethyl sebacate (product name: NIKKOL DES-SP, Nikko Chemicals Co., Ltd.) (20 g) was heated to about 40° C. and leflunomide (2.5 g) was added with stirring. After confirmation of dissolution, α-monoisostearyl glyceryl ether (product name: PENETOL GE-IS, Kao Corporation) (2.5 g) was added with stirring, and the mixture was uniformly dissolved to give a leflunomide solution. Separately, a styrene-isoprene-styrene block copolymer (product name: KRATON D-1107CP, KRATON polymers Japan) (40 g) and a hydrogenated rosin ester derivative (product name: Ester Gum H, Arakawa Chemical Industries, Ltd.) (12.5 g) were mixed with heating. After confirmation of uniform dissolution, diethyl sebacate (product name: NIKKOL DES-SP, Nikko Chemicals Co., Ltd.) (22.5 g) was further added and dissolved. Thereto was added the above-mentioned leflunomide solution to give a 2.5% soluble-type plaster T (adhesive base) of leflunomide.

### Example 21B

Soluble-Type Plaster of Leflunomide (2B)

Diethyl sebacate (product name: NIKKOL DES-SP, Nikko Chemicals Co., Ltd.) (20 g) was heated to about 40° C., and leflunomide (5 g) was added with stirring. After confirmation of dissolution, α-monoisostearyl glyceryl ether (product name: PENETOL GE-IS, Kao Corporation) (2.5 g) was added with stirring and uniformly dissolved to give a leflunomide solution. Separately, a styrene-isoprene-styrene block copolymer (product name: KRATON D-1107CP, KRATON polymers Japan) (40 g) and a hydrogenated rosin ester derivative (product name: Ester Gum H, Arakawa Chemical Industries, Ltd.) (12.5 g) were mixed with heating. After confirmation of uniform dissolution, diethyl sebacate (product name: NIKKOL DES-SP, Nikko Chemicals Co., Ltd.) (20 g) was further added and dissolved. Thereto was added the above-mentioned leflunomide solution, and the mixture was applied to a PET film (single-sided corona discharge treatment, FUJIMORI KOGYO Co., Ltd.) and dried at about 80° C. to give a 5% soluble-type plaster U of leflunomide.

### Example 22B

Suspended Ointment of Leflunomide (1B)

Leflunomide (5 g) was added to plastibase (product name: Plastibase, Bristol Pharmaceuticals Y.K.) (95 g). The mixture was sufficiently stirred at room temperature and defoamed to give a 5% suspended ointment a of leflunomide.

### Example 23B

Suspended Ointment of Leflunomide (2B)

Leflunomide (1 g) was added to plastibase (product name: Plastibase, Bristol Pharmaceuticals Y.K.) (99 g). The mixture was sufficiently stirred at room temperature and defoamed to give a 1% suspended ointment b of leflunomide.

### Example 24B

Suspended Ointment of Leflunomide (3B)

Purified white petrolatum (product name: Sun White P-200, NIKKO RICA CORPORATION) (85 g) and liquid paraffin (YUKA SANGYO CO., LTD.) (10 g) were mixed, and the mixture was heated to about 70° C. to allow melting. The mixture was gradually cooled with stirring and leflunomide (5 g) was added at about 50° C. The mixture was stirred for 10 min and gradually cooled with stirring to solidness to give a 5% suspended ointment c of leflunomide.

### Example 25B

Suspended Ointment of Leflunomide (4B)

Plastibase (product name: Plastibase, Bristol Pharmaceuticals Y. K.) (85 g), α-monoisostearyl glyceryl ether (product name: PENETOL GE-IS, Kao Corporation) (10 g) and leflunomide (5 g) were mixed, sufficiently stirred at room temperature and defoamed to give 5% suspended ointment d of leflunomide.

### Example 26B

Suspended Ointment of Leflunomide (5B)

Purified petrolatum (product name: Sun White P-150, NIKKO RICA CORPORATION) (85 g), isopropyl myristate (product name: NIKKOL IPM-100, Nikko Chemicals Co., Ltd.) (10 g) and leflunomide (5 g) were mixed, and the mixture was sufficiently stirred at room temperature and defoamed to give a 5% suspended ointment e of leflunomide.

### Example 27B

Suspended Liquid of Leflunomide (1B)

Leflunomide (5 g) was added to squalane (product name: Super Squalane, Squatec Co., Ltd.) (95 g), and the mixture was sufficiently stirred at room temperature to give a 5% suspended liquid f of leflunomide.

### Example 28B

Suspended Liquid of Leflunomide (2B) Leflunomide (5 g) was added to liquid paraffin (YUKA SANGYO CO., LTD.) (95 g), and the mixture was sufficiently stirred at room temperature to give a 5% suspended liquid g of leflunomide.

### Comparative Example 1B

Soluble-Type Solution of Leflunomide (17B)

Diethyl sebacate (product name: NIKKOL DES-SP, Nikko Chemicals Co., Ltd.) (35 g) and leflunomide (5 g) were mixed at 40° C. and stirred. Leflunomide was completely dissolved. Liquid paraffin (YUKA SANGYO CO., LTD.) (60 g) was further added with stirring to give a 5% soluble-type solution V of leflunomide.

### Comparative Example 2B

Soluble-Type Solution of Leflunomide (18B)

Diethyl sebacate (product name: NIKKOL DES-SP, Nikko Chemicals Co., Ltd.) (50 g) and leflunomide (5 g) were mixed at 40° C. and stirred to dissolve leflunomide. Liquid paraffin (YUKA SANGYO CO., LTD.) (45 g) was further added with stirring to give a 5% soluble-type solution W of leflunomide.

### Comparative Example 3B

Soluble-Type Solution of Leflunomide (19B)

Diethyl sebacate (product name: NIKKOL DES-SP, Nikko Chemicals Co., Ltd.) (45 g) and leflunomide (5 g) were mixed and stirred at room temperature to dissolve leflunomide. Squalane (50 g) (product name: Super Squalane, Squatec Co., Ltd.) was further added with stirring to give a 5% soluble-type solution X of leflunomide.

### Comparative Example 4B

Suspended Liquid of Leflunomide (3B)

Diethyl sebacate (product name: NIKKOL DES-SP, Nikko Chemicals Co., Ltd.) (40 g) was added to squalane (product name: Super Squalane, Squatec Co., Ltd.) (55 g) and the mixture was homogenized. Leflunomide (5 g) was added and the mixture was sufficiently stirred at room temperature to give a 5% suspended liquid h of leflunomide.

### Comparative Example 5B

Suspended Liquid of Leflunomide (4B)

Diethyl sebacate (product name: NIKKOL DES-SP, Nikko Chemicals Co., Ltd.) (30 g) was added to liquid paraffin (YUKA SANGYO CO., LTD.) (65 g) and the mixture was homogenized. Leflunomide (5 g) was added and the mixture was sufficiently stirred at room temperature to give a 5% suspended liquid i of leflunomide.

### Experimental Example 1A

Treatment Effect of SMP-114 Ointment on Adjuvant Arthritis: (1A)

Using 6-week-old male Lewis rats purchased from Charles River Japan, Inc., this experiment was performed.

Adjuvant arthritis was developed by subcutaneously injecting 0.2 ml of Mycobacterium butyricum dead bacterial cell (Difco) suspended in liquid paraffin at a concentration of 0.5%, into the sole of a right hind foot of rat with ether anesthesia. At day 17 from injection, animals having edema in the left hind foot were selected and grouped based on the volume of the left hind foot and body weight. The test substance was applied once a day for 5 consecutive days starting from the very day of grouping. To prevent the rat from licking the foot, gauze was wound around the external application site. About 5 hr after the final application, the volume of the both hind feet was measure by water substitution method, and difference from the measure at the time of grouping was calculated and evaluated.

The group constitution was as shown in the following Table 1A.

For SMP-114 ointment, one prepared in Example 1A was used, and for preparation blank, one prepared with the same composition but SMP-114 was used.

TABLE 1A
Group constitution in evaluation test

| group | | | | number of |
|---|---|---|---|---|
| No. | test substance | Example | dose^{a)} | animals |
| 1 | blank ointment | — | 100 mg/foot | 7 |
| | preparation | | | |
| 2 | SMP-114 5% | 1A | 100 mg/foot | 7 |
| | ointment (1A) | | | |
| 3 | Inteban ® cream | — | 100 mg/foot | 5 |
| | (commercially | | | |
| | available) | | | |

^{a)}By one administration, 100 mg was applied to one foot and 200 mg to both feet.

The test results are shown in Table 2A. Inteban® cream (Indomethacin 0.75%) used as a positive control was commercially available.

TABLE 2A
Edema suppressive effect of SMP-114 ointment

| | | | increase in hind foot | |
|---|---|---|---|---|
| | | | volume^{a)} | |
| group | | | right hind | left hind |
| No. | test substance | Example | foot (edema) | foot |
| 1 | blank ointment | — | 0.34 ± 0.30 | 0.22 ± 0.24 |
| | preparation | | | |
| 2 | SMP-114 5% | 1A | −0.19 ± 0.34 | −0.25 ± 0.25 |
| | ointment (1A) | | | |
| 3 | Inteban ® cream | — | −1.37 ± 0.35 | −0.80 ± 0.16 |

^{a)}unit amount of increase: ml, mean ± SD

As shown in the results, the SMP-114 5% ointment showed an edema suppressive action by the application once a day.

### Experimental Example 2A

Treatment Effect of SMP-114 Ointment on Adjuvant Arthritis: (2A)

According to Experimental Example 1A, the treatment effect of SMP-114 ointments having various compositions on adjuvant arthritis was evaluated.

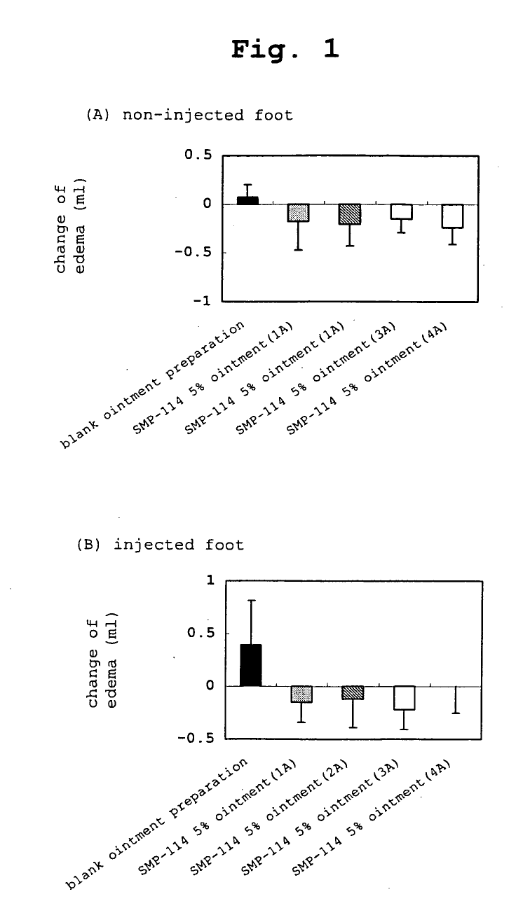

The group constitution was as shown in the following Table 3A. The test results are shown in Table 4A and FIG. 1.

For preparation blank, one prepared having the same composition as Example 1A but SMP-114 was used.

TABLE 3A
Group constitution in evaluation test

| group | test | | | number of |
|---|---|---|---|---|
| No. | substance | Example | dose^{a)} | animal |
| 1 | blank | — | 100 mg/foot | 8 |
| | ointment | | | |
| | preparation | | | |
| 2 | SMP-114 5% | 1A | 100 mg/foot | 8 |
| | ointment (1A) | | | |
| 3 | SMP-114 5% | 4A | 100 mg/foot | 8 |
| | ointment (2A) | | | |
| 4 | SMP-114 5% | 5A | 100 mg/foot | 8 |
| | ointment (3A) | | | |
| 5 | SMP-114 5% | 6A | 100 mg/foot | 8 |
| | ointment (4A) | | | |

^{a)}By one administration, 100 mg was applied to one foot and 200 mg to both feet.

TABLE 4A
Edema suppressive effect of SMP-114 ointment

| | | | increase in hind foot | |
|---|---|---|---|---|
| | | | volume^{a)} | |
| | | | right hind | |
| group | test | | foot | left hind |
| No. | substance | Example | (edema) | foot |
| 1 | blank | — | 0.39 ± 0.42 | 0.07 ± 0.13 |
| | ointment | | | |
| | preparation | | | |
| 2 | SMP-114 5% | 1A | −0.15 ± 0.19 | −0.18 ± 0.29 |
| | ointment (1A) | | | |
| 3 | SMP-114 5% | 4A | −0.12 ± 0.27 | −0.21 ± 0.22 |
| | ointment (2A) | | | |
| 4 | SMP-114 5% | 5A | −0.22 ± 0.19 | −0.15 ± 0.14 |
| | ointment (3A) | | | |
| 5 | SMP-114 5% | 6A | 0.00 ± 0.25 | −0.24 ± 0.17 |
| | ointment (4A) | | | |

^{a)}unit of increased amount: ml, mean ± SD

As shown in the results, the SMP-114 5% ointment showed an edema suppressive action by the application once a day.

From the above results, it became clear that SMP-114 showed efficacy on external application.

### Experimental Example 3A

Treatment Effect of SMP-114 Suspension/Solution on Adjuvant Arthritis

According to Experimental Example 1A, the treatment effect of SMP-114 suspensions having various compositions on adjuvant arthritis was evaluated.

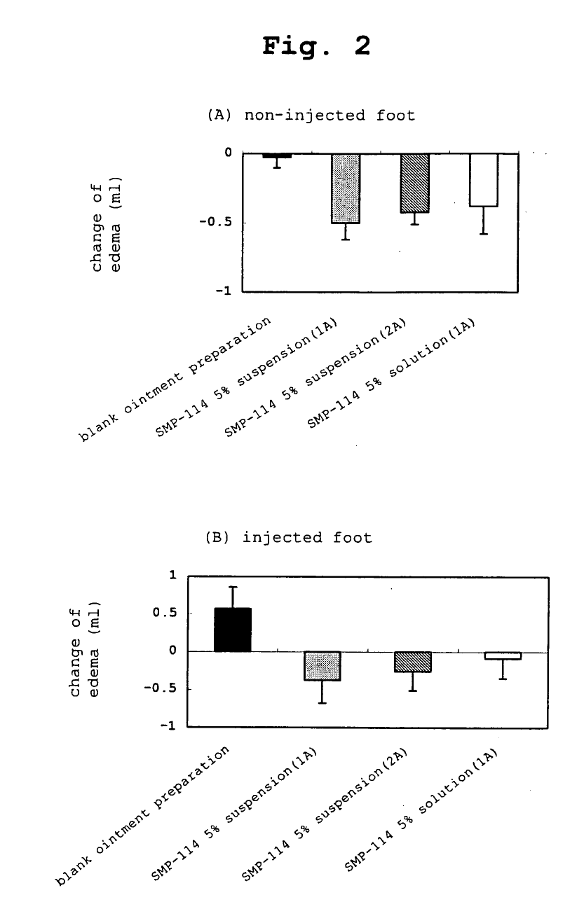

The group constitution was as shown in the following Table 5A. The test results are shown in Table 6A and FIG. 2.

For preparation blank, one prepared having the same composition as Example 1A but SMP-114 was used.

TABLE 5A
Group constitution in evaluation test

| group | test | | | number of |
|---|---|---|---|---|
| No. | substance | Example | dose^{a)} | animal |
| 1 | blank | — | 100 mg/foot | 7 |
| | ointment | | | |
| | preparation | | | |
| 2 | SMP-114 5% | 7A | 100 mg/foot | 7 |
| | suspension | | | |
| | (1A) | | | |
| 3 | SMP-114 5% | 8A | 100 mg/foot | 7 |
| | suspension | | | |
| | (2A) | | | |
| 4 | SMP-114 5% | 9A | 100 mg/foot | 7 |
| | solution (1A) | | | |

^{a)}By one administration, 100 mg was applied to one foot and 200 mg to both feet.

TABLE 6A
Edema suppressive effect of SMP-114
suspension/solution

| | | | increase in hind foot | |
|---|---|---|---|---|
| | | | volume^{a)} | |
| group | test | | right hind | left hind |
| No. | substance | Example | foot (edema) | foot |
| 1 | blank | — | 0.57 ± 0.29 | −0.03 ± 0.07 |
| | ointment | | | |
| | preparation | | | |
| 2 | SMP-114 5% | 7A | −0.38 ± 0.30 | −0.50 ± 0.12 |
| | suspension | | | |
| | (1A) | | | |
| 3 | SMP-114 5% | 8A | −0.26 ± 0.25 | −0.42 ± 0.09 |
| | suspension | | | |
| | (2A) | | | |
| 4 | SMP-114 5% | 9A | −0.09 ± 0.26 | −0.38 ± 0.20 |
| | solution (3A) | | | |

^{a)}unit of increased amount: ml, average ± SD

As shown in the results, the SMP-114 5% suspension/solution showed an edema suppressive action by the application once a day.

From the above results, it became clear that SMP-114 showed efficacy on external application.

### Experimental Example 4A

Treatment Effect of Leflunomide Suspension on Adjuvant Arthritis

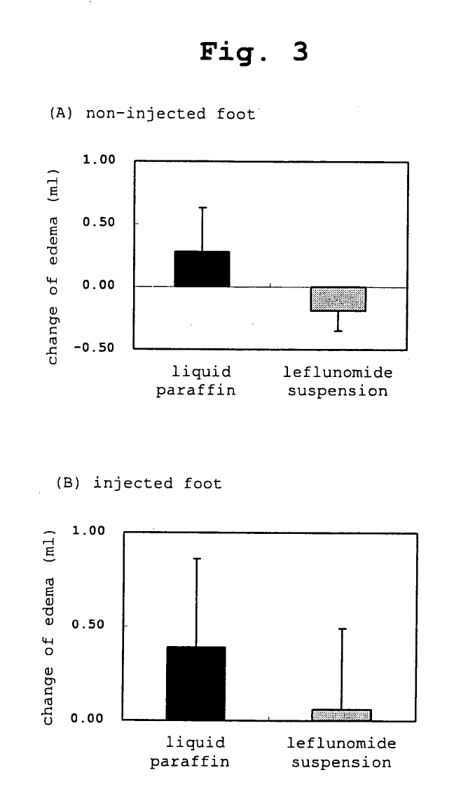

Using the same rat adjuvant arthritis models as in Experimental Example 1A, leflunomide suspension prepared in Example 10A was applied for 5 consecutive days by 100 μl per one foot once a day. The increase or decrease in the swelling of the joint before and after the application period was observed. The amount of the 5% suspension applied corresponds to the dose of 50 mg/kg body weight of the rat. The results are shown in Table 7A and FIG. 3. The liquid paraffin used as the negative control was commercially available.

TABLE 7A
Edema suppressive effect of leflunomide suspension

| | | | increase in hind foot | |
|---|---|---|---|---|
| | | | volume^{a)} | |
| | | | right hind | |
| group | test | | foot | left hind |
| No. | substance | Example | (edema) | foot |
| 1 | liquid | — | 0.39 ± 0.47 | 0.28 ± 0.35 |
| | paraffin | | | |
| 2 | leflunomide | 10A | 0.06 ± 0.43 | −0.19 ± 0.16 |
| | 5% suspension | | | |

^{a)}unit of increased amount: ml, mean ± SD

As shown in the results, the leflunomide 5% suspension showed an edema suppressive action by the application once a day.

### Experimental Example 5A

Treatment Effect of Leflunomide Ointment on Adjuvant Arthritis

According to Experimental Example 1A, the treatment effect of leflunomide ointments having various contents on adjuvant arthritis was evaluated.

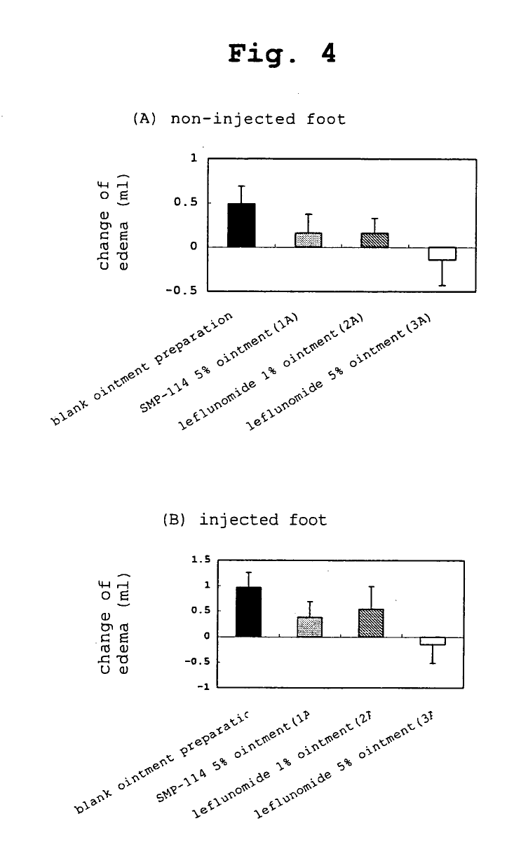

The group constitution was as shown in the following Table 8A. The test results are shown in Table 9A and FIG. 4.

For preparation blank, one prepared having the same composition as Example 1A but SMP-114 was used.

TABLE 8A
Group constitution in evaluation test

| group | | | | number of |
|---|---|---|---|---|
| No. | Test substance | Example | dose^{a)} | animal |
| 1 | blank ointment | — | 100 mg/foot | 8 |
| | preparation | | | |
| 2 | SMP-114 5% | 1A | 100 mg/foot | 8 |
| | ointment (1A) | | | |
| 3 | leflunomide 1% | 12A | 100 mg/foot | 8 |
| | ointment (2A) | | | |
| 4 | leflunomide 5% | 13A | 100 mg/foot | 8 |
| | ointment (3A) | | | |

^{a)}By one administration, 100 mg was applied to one foot and 200 mg to both feet.

TABLE 9A
Edema suppressive effect of leflunomide ointment

| | | | increase in hind foot | |
|---|---|---|---|---|
| | | | volume^{a)} | |
| | | | right hind | |
| group | | | foot | left hind |
| No. | Test substance | Example | (edema) | foot |
| 1 | blank ointment | — | 0.97 ± 0.29 | 0.49 ± 0.20 |
| | preparation | | | |
| 2 | SMP-114 5% | 1A | 0.38 ± 0.32 | 0.16 ± 0.21 |
| | ointment (1A) | | | |
| 3 | leflunomide 1% | 12A | 0.54 ± 0.45 | 0.16 ± 0.17 |
| | ointment (2A) | | | |
| 4 | leflunomide 5% | 13A | −0.15 ± 0.36 | −0.14 ± 0.29 |
| | ointment (3A) | | | |

^{a)}unit of increased amount: ml, mean ± SD

As shown in the results, all leflunomide ointments showed an edema suppressive action by the application once a day.

From the above results, it became clear that leflunomide showed efficacy on external application.

### Experimental Example 1B

Treatment Effect of a Dosage Form for External Application of Leflunomide on Adjuvant Arthritis—(1B)

Using 6-week-old male Lewis rats purchased from Charles River Japan, Inc., this experiment was performed.

Adjuvant arthritis was developed by subcutaneously injecting 0.2 ml of Mycobacterium butyricum dead bacterial cell (Difco) suspended in liquid paraffin at a concentration of 0.5%, into the sole of a right hind foot of rat with ether anesthesia. At day 17 from injection, animals having edema in the left hind foot were selected and grouped based on the volume of the left hind foot and body weight. The test substance was applied once a day for 5 consecutive days starting from the very day of grouping. To prevent the rat from licking the foot, aluminum foil was wound around the external application site. About 5 hr after the final application, the volume of the both hind feet was measure by water substitution method, and difference from the measure at the time of grouping was calculated and evaluated.

The group constitution was as shown in the following Table 1B.

For a dosage form for external application of leflunomide, one prepared in Example was used, and as preparation blank for each sample, one prepared with the same composition but leflunomide was used.

TABLE 1B
Group constitution in evaluation test

| group | | | | number of |
|---|---|---|---|---|
| No. | Test substance | Example | dose^{a)} | animal |
| 1 | blank soluble-type | — | 100 mg/foot | 6 |
| | solution A | | | |
| 2 | leflunomide 1% | Example 1B | 100 mg/foot | 6 |
| | soluble-type | | | |
| | solution A | | | |
| 3 | leflunomide 5% | Example 2B | 100 mg/foot | 6 |
| | soluble-type | | | |
| | solution B | | | |
| 4 | blank soluble-type | — | 100 mg/foot | 6 |
| | solution D | | | |
| 5 | leflunomide 5% | Example 4B | 100 mg/foot | 6 |
| | soluble-type | | | |
| | solution D | | | |
| 6 | blank ointment a | — | 100 mg/foot | 6 |
| 7 | leflunomide 5% | Example 22B | 100 mg/foot | 6 |
| | suspended ointment a | | | |

^{a)}By one administration, 100 mg was applied to one foot and 200 mg to both feet.

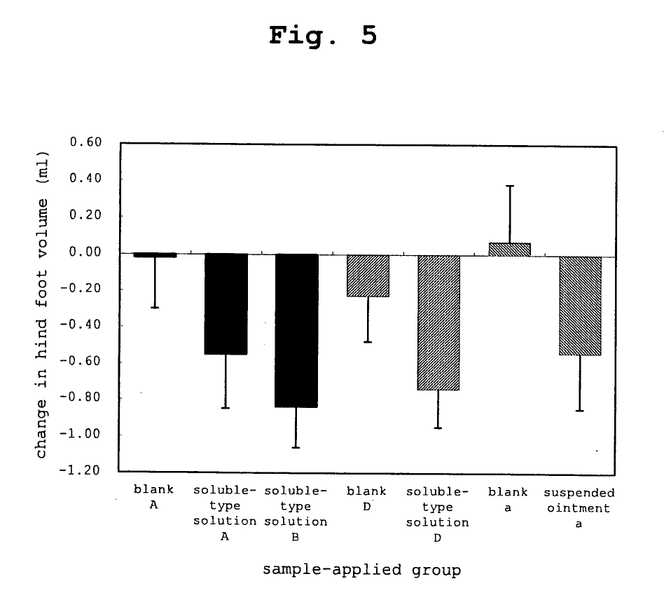

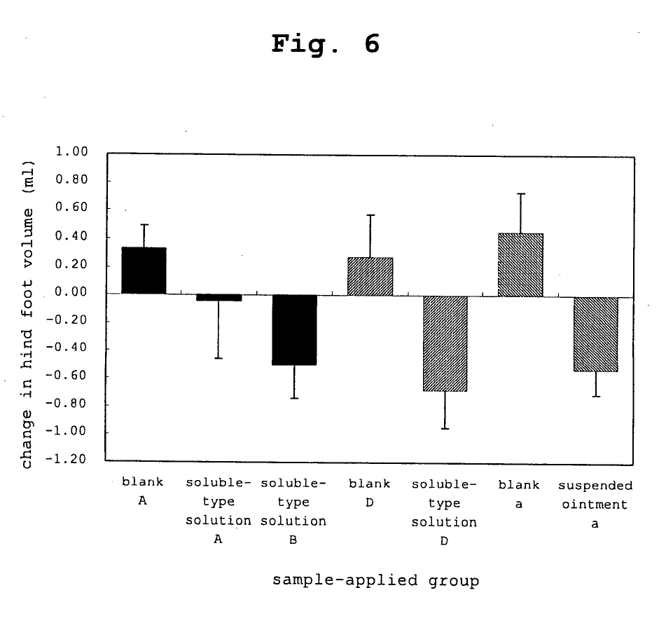

The test results are shown in Table 2B and FIGS. 5, 6.

TABLE 2B
Edema suppressive effect of a dosage form for external
application of leflunomide

| | | increase in hind foot | |
|---|---|---|---|
| | | volume^{a)} | |
| group | | right hind | left hind |
| No. | Test substance | foot (edema) | foot |
| 1 | blank soluble-type | 0.33 ± 0.16 | −0.02 ± 0.28 |
| | solution A | | |
| 2 | leflunomide 1% soluble- | −0.04 ± 0.42 | −0.55 ± 0.30 |
| | type solution A | | |
| 3 | leflunomide 5% soluble- | −0.50 ± 0.24 | −0.84 ± 0.22 |
| | type solution B | | |
| 4 | blank soluble-type | 0.27 ± 0.30 | −0.23 ± 0.25 |
| | solution D | | |
| 5 | leflunomide 5% soluble- | −0.68 ± 0.27 | −0.74 ± 0.21 |
| | type solution D | | |
| 6 | blank ointment a | 0.45 ± 0.28 | 0.07 ± 0.31 |
| 7 | leflunomide 5% suspended | −0.53 ± 0.18 | −0.54 ± 0.31 |
| | ointment a | | |

^{a)}unit amount of increase: ml, average ± SD

As shown in the results, the soluble-type solution and the suspended ointment of leflunomide showed a high edema suppressive effect as compared to the blank, by the application thereof once a day.

In general, for evaluation of an antirheumatic effect using an adjuvant arthritis model, the suppressive effect on the non-injected foot is more significant than the effect on the injected foot (edema).

The results of Table 2B reveal the high edema suppressive effect of the leflunomide-containing preparation of the present invention also on the non-injected foot.

### Experimental Example 2B

Treatment Effect of a Dosage Form for External Application of Leflunomide on Adjuvant Arthritis—(2B)

According to Experimental Example 1B, the treatment effect of dosage forms for external application of leflunomide having various compositions on adjuvant arthritis was evaluated.

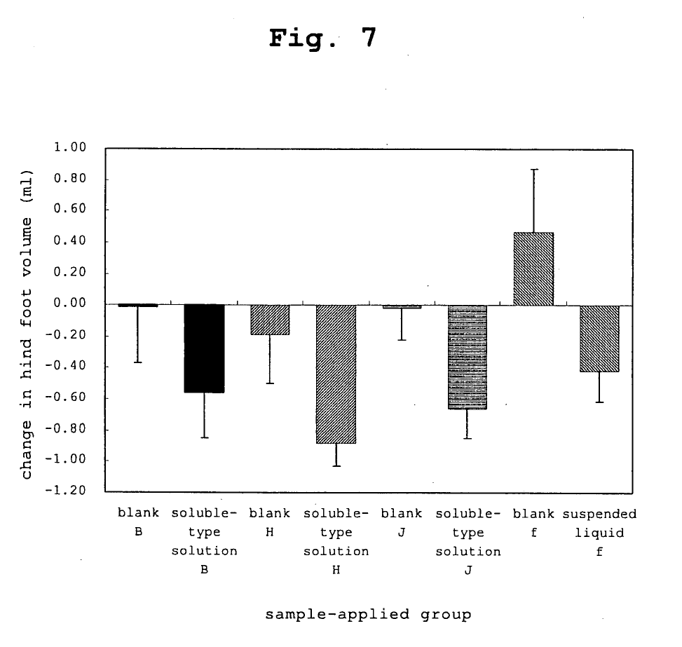

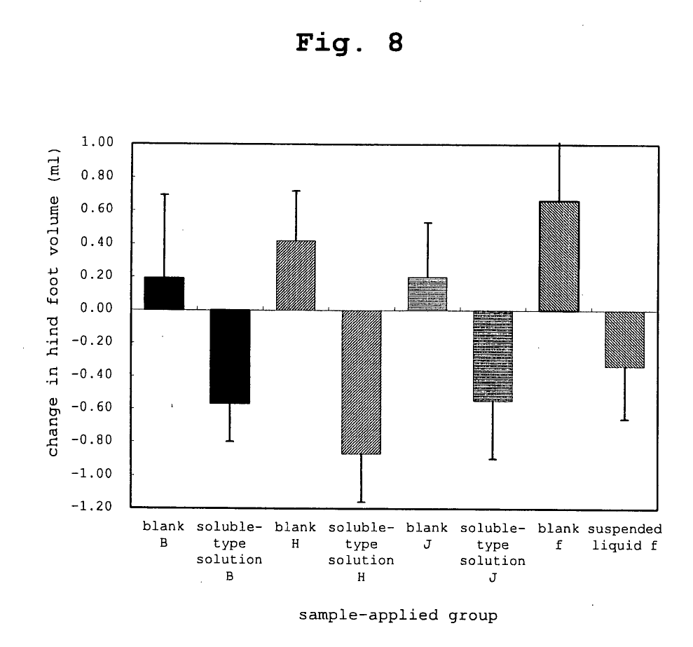

The group constitution was as shown in the following Table 3B. The test results are shown in Table 4B and FIGS. 7 and 8.

As the preparation blank that was accorded a symbol corresponding to each sample, one having the same composition but leflunomide was used.

TABLE 3B
Group constitution in evaluation test

| group | | | | number of |
|---|---|---|---|---|
| No. | Test substance | Example | dose^{a)} | animal |
| 1 | blank soluble-type | — | 100 mg/foot | 8 |
| | solution B | | | |
| 2 | leflunomide 5% | Example | 100 mg/foot | 8 |
| | soluble-type | 2B | | |
| | solution B | | | |
| 3 | blank soluble-type | — | 100 mg/foot | 8 |
| | solution H | | | |
| 4 | leflunomide 5% | Example | 100 mg/foot | 8 |
| | soluble-type | 8B | | |
| | solution H | | | |
| 5 | blank soluble-type | — | 100 mg/foot | 8 |
| | solution J | | | |
| 6 | leflunomide 5% | Example | 100 mg/foot | 8 |
| | soluble-type | 10B | | |
| | solution J | | | |
| 7 | blank suspended | — | 100 mg/foot | 8 |
| | liquid f | | | |
| 8 | Leflunomide 5% | Example | 100 mg/foot | 8 |
| | suspended liquid f | 27B | | |

^{a)}By one administration, 100 mg was applied to one foot and 200 mg to both feet.

TABLE 4B
Edema suppressive effect of a dosage form for external
application of leflunomide

| | | increase in hind foot | |
|---|---|---|---|
| | | volume^{a)} | |
| group | | right hind | left hind |
| No. | Test substance | foot (edema) | foot |
| 1 | Soluble-type solution | 0.19 ± 0.50 | −0.01 ± 0.36 |
| | blank B | | |
| 2 | leflunomide 5% soluble- | −0.57 ± 0.23 | −0.56 ± 0.29 |
| | type solution B | | |
| 3 | Soluble-type solution | 0.42 ± 0.30 | −0.19 ± 0.31 |
| | blank H | | |
| 4 | leflunomide 5% soluble- | −0.87 ± 0.29 | −0.88 ± 0.15 |
| | type solution H | | |
| 5 | Soluble-type solution | 0.20 ± 0.33 | −0.02 ± 0.20 |
| | blank J | | |
| 6 | leflunomide 5% soluble- | −0.55 ± 0.35 | −0.66 ± 0.19 |
| | type solution J | | |
| 7 | Suspended liquid blank f | 0.66 ± 0.37 | 0.47 ± 0.40 |
| 8 | leflunomide 5% suspended | −0.34 ± 0.32 | −0.42 ± 0.19 |
| | liquid f | | |

^{a)}unit amount of increase: ml, mean ± SD

As shown in the results, all leflunomide-containing preparations of the present invention showed an edema suppressive effect, by the application thereof once a day.

When α-monoalkyl glyceryl ether was added, the edema suppressive effect was strikingly enhanced as compared to the formulation without the addition.

From the above results, leflunomide shows superior efficacy when transdermally administered as contained in a particular base.

### Experimental Example 3B

Treatment Effect of Leflunomide Soluble-Type Solution on Adjuvant Arthritis—(3B)

According to Experimental Example 1B, the treatment effect of a dosage form for external application of leflunomide having various compositions on adjuvant arthritis was evaluated.

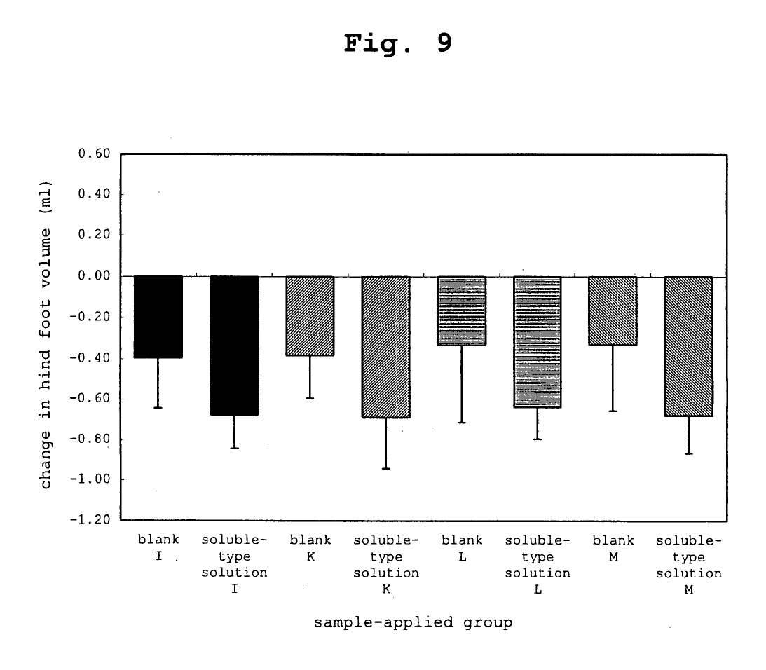

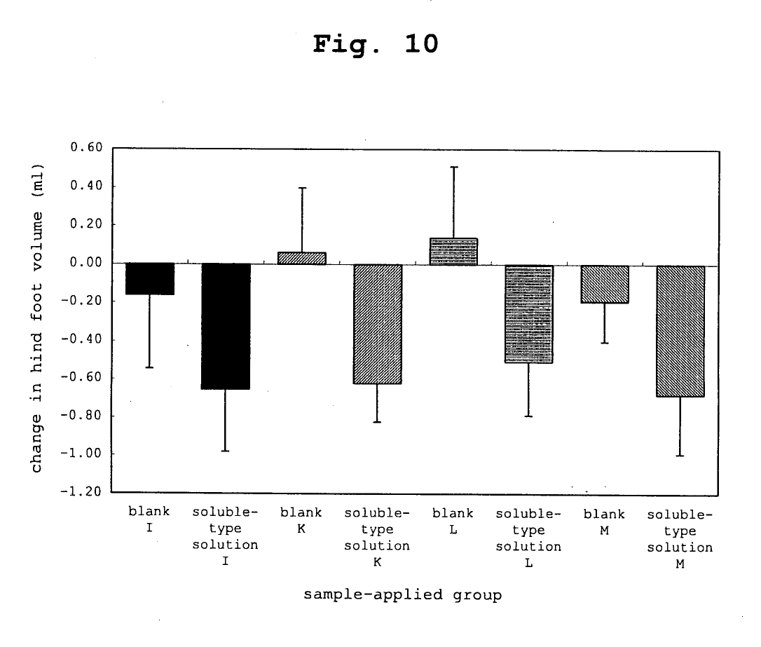

The group constitution was as shown in the following Table 3B. The test results are shown in Table 5B and FIGS. 9 and 10.

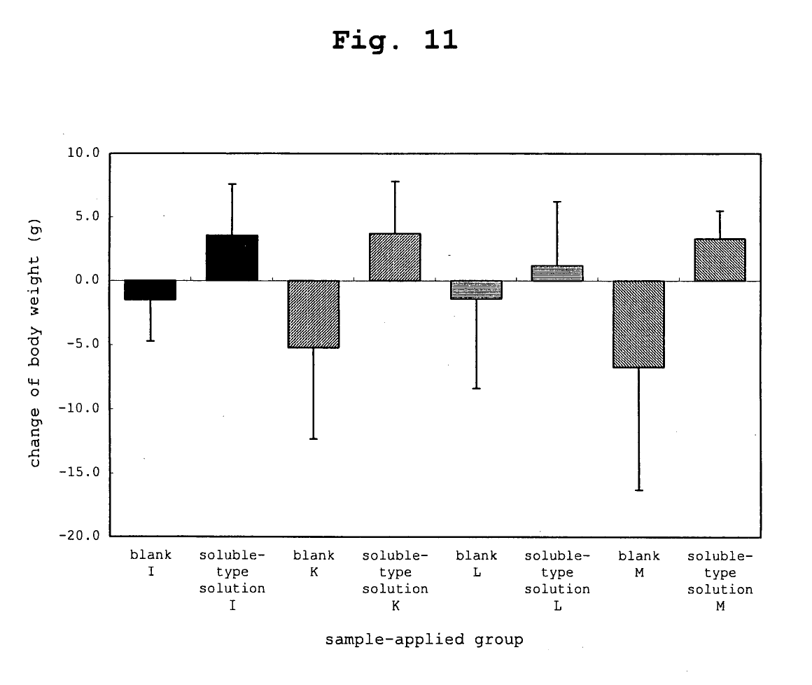

The changes in the body weight before and after the test are shown in Table 7B and FIG. 11.

As the preparation blank that was accorded a symbol corresponding to each sample, one having the same composition but leflunomide was used.

TABLE 5B
Group constitution in evaluation test

| group | | | | number of |
|---|---|---|---|---|
| No. | Test substance | Example | dose^{a)} | animal |
| 1 | Blank soluble-type | — | 100 mg/foot | 7 |
| | preparation I | | | |
| 2 | leflunomide 5% | Example | 100 mg/foot | 7 |
| | soluble-type | 9B | | |
| | solution I | | | |
| 3 | blank soluble-type | — | 100 mg/foot | 7 |
| | preparation K | | | |
| 4 | leflunomide 2.5% | Example | 100 mg/foot | 7 |
| | soluble-type | 11B | | |
| | solution K | | | |
| 5 | blank soluble-type | — | 100 mg/foot | 7 |
| | preparation L | | | |
| 6 | leflunomide 2.5% | Example | 100 mg/foot | 7 |
| | soluble-type | 12B | | |
| | solution L | | | |
| 7 | blank soluble-type | — | 100 mg/foot | 7 |
| | preparation M | | | |
| 8 | leflunomide 2.5% | Example | 100 mg/foot | 7 |
| | soluble-type | 13B | | |
| | solution M | | | |

^{a)}By one administration, 100 mg was applied to one foot and 200 mg to both feet.

TABLE 6B
Edema suppressive effect of a dosage form for external
application of leflunomide

| | | increase in hind foot | |
|---|---|---|---|
| | | volume^{a)} | |
| group | | right hind | left hind |
| No. | Test substance | foot (edema) | foot |
| 1 | blank soluble-type | −0.16 ± 0.38 | −0.40 ± 0.25 |
| | preparation I | | |
| 2 | leflunomide 5% soluble- | −0.65 ± 0.33 | −0.68 ± 0.16 |
| | type solution I | | |
| 3 | blank soluble-type | 0.06 ± 0.34 | −0.38 ± 0.21 |
| | preparation K | | |
| 4 | leflunomide 2.5% soluble- | −0.62 ± 0.20 | −0.69 ± 0.25 |
| | type solution K | | |
| 5 | blank soluble-type | 0.14 ± 0.37 | −0.33 ± 0.38 |
| | preparation L | | |
| 6 | leflunomide 2.5% soluble- | −0.51 ± 0.28 | −0.64 ± 0.16 |
| | type solution L | | |
| 7 | blank soluble-type | −0.19 ± 0.21 | −0.33 ± 0.33 |
| | preparation M | | |
| 8 | leflunomide 2.5% soluble- | −0.68 ± 0.31 | −0.68 ± 0.19 |
| | type solution M | | |

^{a)}unit amount of increase: ml, mean ± SD

As shown in the results, all soluble-type solutions of leflunomide of the present invention showed a high edema suppressive effect as compared to the blank, by the application thereof once a day.

TABLE 7B
Effect of dosage form for external application of
leflunomide on body weight gain

| group | | body weight |
|---|---|---|
| No. | test substance | gain^{a)} |
| 1 | blank soluble-type | −1.5 ± 3.2 |
| | preparation I | |
| 2 | leflunomide 5% soluble- | 3.5 ± 4.0 |
| | type solution I | |
| 3 | blank soluble-type | −5.2 ± 7.1 |
| | preparation K | |
| 4 | leflunomide 2.5% soluble- | 3.7 ± 4.1 |
| | type solution K | |
| 5 | blank soluble-type | −1.4 ± 7.0 |
| | preparation L | |
| 6 | leflunomide 2.5% soluble- | 1.2 ± 5.0 |
| | type solution L | |
| 7 | blank soluble-type | −6.7 ± 9.5 |
| | preparation M | |
| 8 | leflunomide 2.5% soluble- | 3.3 ± 2.2 |
| | type solution M | |

^{a)}unit amount of increase: ml, mean ± SD

As shown in the results, the preparation blank showed a decrease in the body weight. However, all leflunomide soluble-type solutions showed an increase in the body weight. Decrease in the body weight is known to be one of the side effects of leflunomide.

From the above results, it is clear that the leflunomide-containing preparation of the present invention not only shows superior efficacy when contained in a dissolution state in a particular base, but also is superior in safety.

### Experimental Example 4B

Treatment Effect of Leflunomide Soluble-Type Solution on Adjuvant Arthritis—(4B)

According to Experimental Example 1B, the treatment effect of leflunomide soluble-type solution and leflunomide soluble-type plaster having various compositions on adjuvant arthritis was evaluated. Plaster U was adhered to cover the entire hind foot. To prevent dislocation of the plaster, the plaster was fixed with a bandage. Since complete adhesion of plaster T to the rat hind foot is difficult to achieve, an adhesive base alone without a backing layer was administered.

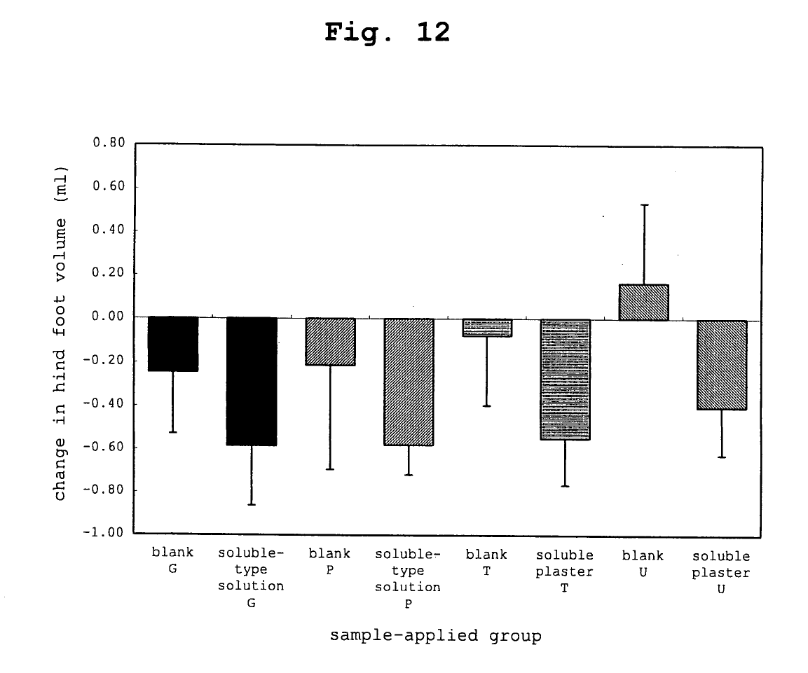

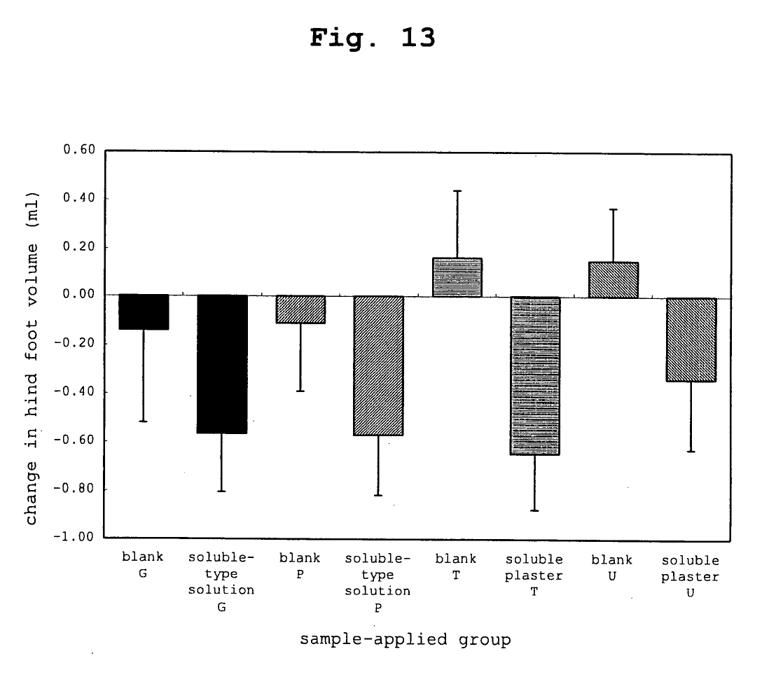

The group constitution was as shown in the following Table 8B. The test results are shown in Table 9B and FIGS. 12 and 13.

As the preparation blank that was accorded a symbol corresponding to each sample, one having the same composition but leflunomide was used.

TABLE 8B
Group constitution in evaluation test

| group | | | | number of |
|---|---|---|---|---|
| No. | Test substance | Example | dose | animal |
| 1 | blank soluble-type | — | 100 mg/foot^{a)} | 8 |
| | solution G | | | |
| 2 | leflunomide 2.5% | Example | 100 mg/foot^{a)} | 8 |
| | soluble-type solution G | 7B | | |
| 3 | blank soluble-type | — | 100 mg/foot^{a)} | 8 |
| | solution p | | | |
| 4 | leflunomide 2.5% | Example | 100 mg/foot^{a)} | 8 |
| | soluble-type solution P | 16B | | |
| 5 | blank plaster | — | 100 mg/foot^{b)} | 8 |
| | T(adhesive base) | | | |
| 6 | leflunomide 2.5% | Example | 100 mg/foot^{b)} | 8 |
| | soluble-type plaster | 20B | | |
| | T(adhesive base) | | | |
| 7 | blank plaster U | — | 50 mg/foot^{c)} | 8 |
| 8 | leflunomide 5% | Example | 50 mg/foot^{c)} | 8 |
| | soluble-type plaster U | 21B | | |

^{a)}By one administration, 100 mg was applied to one foot and 200 mg to both feet.
^{b)}By one administration, 100 mg of adhesive base was applied to one foot and 200 mg thereof to both feet.
^{c)}By one administration, 50 mg of adhesive base was applied to one foot and 100 mg thereof to both feet.

TABLE 9B
Edema suppressive effect of a dosage form for
external application of leflunomide

| | | increase in hind foot | |
|---|---|---|---|
| | | volume^{a)} | |
| group | | right hind foot | left hind |
| No. | Test substance | (edema) | foot |
| 1 | blank soluble-type solution G | −0.14 ± 0.38 | −0.25 ± 0.28 |
| 2 | leflunomide 2.5% soluble-type | −0.57 ± 0.24 | −0.58 ± 0.28 |
| | solution G | | |
| 3 | blank soluble-type solution P | −0.11 ± 0.28 | −0.12 ± 0.48 |
| 4 | leflunomide 2.5% soluble-type | −0.57 ± 0.25 | −0.58 ± 0.14 |
| | solution P | | |
| 5 | blank plaster T (adhesive base) | 0.16 ± 0.28 | −0.08 ± 0.32 |
| 6 | leflunomide 2.5% soluble-type | −0.65 ± 0.23 | −0.55 ± 0.22 |
| | plaster T (adhesive base) | | |
| 7 | blank plaster U | 0.15 ± 0.22 | 0.16 ± 0.37 |
| 8 | leflunomide 5% soluble-type | −0.34 ± 0.29 | −0.41 ± 0.22 |
| | plaster U | | |

^{a)}unit amount of increase: ml, mean ± SD

As shown in the results, all soluble-type preparations of the present invention showed a high edema suppressive effect as compared to the blank, by the application thereof once a day.

Although plaster U could not adhered completely, it showed a sufficient edema suppressive effect as compared to the blank.

### Experimental Example 5B

Measurement of Blood Concentration by Transdermal Administration of Leflunomide Soluble-Type Solution

The dosage form for external application of leflunomide as shown in Example was administered once to adjuvant arthritis rats prepared in the same manner as in Experimental Example 1B and the concentration of leflunomide in serum was measured using LC-MS/MS. In addition, the concentration in serum on simultaneous oral administration of leflunomide once (20 mg/kg) was measured and compared.

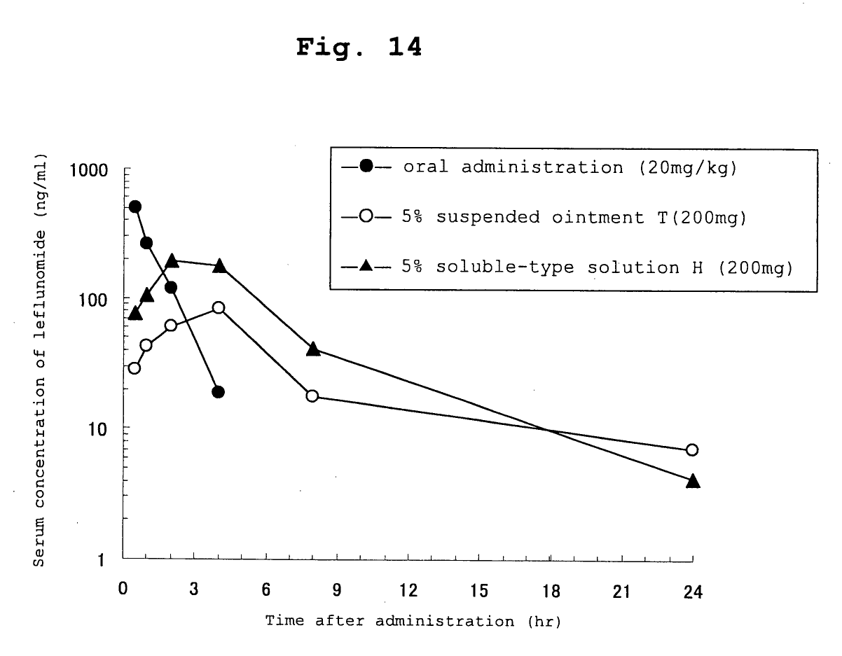

The group constitution is shown in Table 10B, and the results are shown in Table 11B and FIG. 14.

TABLE 10B
Group constitution in evaluation test

| | | | | | number |
|---|---|---|---|---|---|
| group | | | | | of |
| No. | Test substance | Example | dose | | animal |
| 1 | leflunomide 5% | Example | 100 | mg/foot^{a)} | 3 |
| | soluble-type solution H | 8B | | | |
| 2 | leflunomide 5% | Example | 100 | mg/foot^{a)} | 3 |
| | suspended ointment a | 22B | | | |
| 3 | leflunomide (oral | — | 20 | mg/kg oral | 3 |
| | administration) | | | | |

^{a)}By one administration, 100 mg was applied to one foot and 200 mg to both feet.

TABLE 11B
Drug kinetics parameter in rats with adjuvant arthritis

| group | | AUC^{a)} | Cmax^{b)} | tmax^{c)} |
|---|---|---|---|---|
| No. | Test substance | ng · hr/mL | ng/mL | hr |
| 1 | leflunomide 5% soluble-type | 1248 | 192 | 2 |
| | solution H | | | |
| 2 | leflunomide 5% suspended | 573 | 83.5 | 4 |
| | ointment a | | | |
| 3 | leflunomide (oral administration) | 600 | 502 | 0.5 |

^{a)}area under the drug concentration-time curve [AUC^{(0-T)}]
^{b)}maximum drug concentration
^{c)}times to reach maximum drug concentration

As shown in the results, the leflunomide soluble-type solution showed Cmax suppressed to not more than ½ as compared to oral administration, but showed tmax that increased to 4 times higher value and AUC^{(0-T)} that increased to about 2 times higher value. In addition, the leflunomide suspended ointment showed almost the same AUC^{(0-T)} as compared to oral administration but Cmax suppressed to not more than ⅕ and tmax improved 8 times.

From the above results, the pharmaceutical composition for transdermal administration of the present invention is clearly a superior preparation from the aspects of side effect and durability.

### Experimental Example 6B

Stability Evaluation of Soluble-Type Solution of Leflunomide-(1B)

Each preparation prepared in Examples 2B, 6B, 8B and 12B was subjected to a stability test under severe conditions.

To be specific, each of the above-mentioned samples was placed in a 10 mL glass screw-in test tube (NR-10, Maruemu Co., Ltd.) by 3 mL and sealed. The tube was preserved in a constant-temperature bath at 60° C., taken out after 3 weeks and 6 weeks of preservation and subjected to a liquid chromatography analysis.

The stability was evaluated by simultaneously analyzing a separately prepared leflunomide standard solution and comparing a related substance content (%), which is the total of impurity peak area of each sample based on the leflunomide peak area of the standard solution as 100, with the initial value. The related substance mass contains a peak due to the base.

The results are shown in Table 12B.

TABLE 12B
Stability of soluble-type solution of leflunomide

| | related substance content (%) | | |
|---|---|---|---|
| sample | initial | 60° C. × 3 weeks | 60° C. × 6 weeks |
| Example 2B | 0.32 | 0.31 | 0.29 |
| Example 6B | 0.28 | 0.28 | 0.30 |
| Example 8B | 0.35 | 0.32 | 0.30 |
| Example 12B | 0.26 | 0.22 | 0.25 |

As is clear from Table 12B, all the soluble-type pharmaceutical compositions for transdermal administration of the present invention show high stability.

### Experimental Example 7B

Stability Evaluation of Soluble-Type Solution of Leflunomide—(2B)

The soluble-type solutions A-P prepared in Examples 1B-16B and soluble-type solutions V-X prepared in Comparative Examples 1B-3B were each placed in a 10 mL glass screw-in test tube (NR-10, Maruemu Co., Ltd.), sealed and preserved in a refrigerator at 5° C. The test tubes were taken out at a given time after 1, 3, 5, 7, 14, 21 and 28 days of preservation. After allowing to stand at room temperature for 2 hr, the presence of crystal precipitation was visually observed. As a result, soluble-type solutions A-P prepared in Examples 1B-16B did not show precipitation of a solid such as crystal and the like.

In contrast, the soluble-type solutions V, W and X prepared in Comparative Examples 1B to 3B showed crystal precipitation and phase separation of leflunomide within 3 days by a similar test.

From this result, it was confirmed that the soluble-type pharmaceutical composition for transdermal administration of the present invention stably dissolved the active ingredient in a carrier for transdermal administration.

### Experimental Example 8B

Stability Evaluation of Leflunomide Suspended Ointment—(1B)

The preparations (5% leflunomide suspended ointment a, 1% leflunomide suspended ointment b) prepared in Examples 22B and 23B were subjected to a severe stability test, and the stability of the active ingredient was evaluated.

To be specific, a sample (10 g) was filled in an aluminum tube (Kansai Tube Co., Ltd.), sealed and preserved in a thermostatic tank at 60° C. After 2 weeks or 4 weeks of preservation, the tubes were taken out and subjected to a liquid chromatography analysis. For the stability, the total of the proportion of impurity peak relative to leflunomide peak area is taken as the related substance content, and compared with the initial value. The test was conducted with n=3, and the average value was calculated.

The results are shown in Table 13B.

TABLE 13B
Severe stability of leflunomide suspended ointment

| | average related substance content (%) | | |
|---|---|---|---|
| sample | Initial | 60° C. × 2 weeks | 60° C. × 4 weeks |
| Example 22B | 0.18 | 0.19 | 0.19 |
| Example 23B | 0.18 | 0.19 | 0.19 |

As a result of the severe stability test at 60° C., the suspended-type pharmaceutical composition for transdermal administration of the present invention showed high stability, as is clear from Table 13B.

The particle size of the crystals suspended in a carrier in each sample did not change even after the lapse of 6 weeks at 60° C.

### Experimental Example 9B

Stability Evaluation of Leflunomide Suspended Ointment—(2B)

The preparations (5% leflunomide suspended ointment a, 1% leflunomide suspended ointment B) prepared in Examples 22B and 23B were subjected to a long-term preservation stability test and the stability of the active ingredient was evaluated.

To be specific, a sample (10 g) was filled in an aluminum tube (Kansai Tube Co., Ltd.), sealed and preserved in a refrigerator at 5° C. After 4 months or 6 months of preservation, the tubes were taken out and subjected to a liquid chromatography analysis. For the stability, the total of the proportion of impurity peak relative to leflunomide peak area is taken as the related substance content, and compared with the initial value. The test was conducted with n=3, and the average value was calculated.

The results are shown in Table 14B.

TABLE 14B
long-term stability of leflunomide suspended ointment

| | average related substance content (%) | | |
|---|---|---|---|
| sample | initial | 5° C. × 4 months | 5° C. × 6 months |
| Example 22B | 0.18 | 0.19 | 0.19 |
| Example 23B | 0.18 | 0.19 | 0.19 |

As a result of the long-term stability test at 5° C., the suspended-type pharmaceutical composition for transdermal administration of the present invention showed high stability, as is clear from Table 14B.

The particle size of the crystals suspended in each sample did not change even after the lapse of 6 months at 50° C.

### Experimental Example 10B

Stability Evaluation of Leflunomide Suspended-Type Liquid

Using the liquids prepared in Examples 27B and 28B and Comparative Examples 4B and 5B, a stability test of crystal form was performed.

To be specific, each sample was filled in a 10 mL glass covered test tube, sealed, warmed in a water bath at 40° C. with shaking and taken out after 6 hr. The leflunomide crystals precipitated in the test tube was visually observed. As a result, the samples of Examples 27 and 28 clearly showed decreased crystal amount as compared to the samples of Comparative Examples 4 and 5.

Then, the samples were preserved in a refrigerator at 5° C. for 3 days. The samples were taken out, observed for the shape of the suspended crystals with a light microscope, and compared with a sample separately prepared and preserved at 5° C. immediately thereafter.

As a result, the liquids prepared in Examples 27B and 28B did not show any change in the crystal shape but the liquids prepared in Comparative Examples 4B and 5B showed a number of crystals having the size of not less than 100 μm, thus confirming clear changes in the crystal shape.

From the above results, the suspended-type pharmaceutical composition for transdermal administration of the present invention is clearly a preparation superior in the stability.

### Experimental Example 11B

Treatment Effect of Leflunomide Suspended-Type Ointment on Adjuvant Arthritis

Using 6-week-old male Lewis rats purchased from Charles River Japan, Inc., this experiment was performed.

Adjuvant arthritis was developed by subcutaneously injecting 0.2 ml of Mycobacterium butyricum dead bacterial cell (Difco) suspended in liquid paraffin at a concentration of 0.5%, into the sole of a right hind foot of rat with ether anesthesia. At day 17 from injection, animals having edema in the left hind foot were selected and grouped based on the volume of the left hind foot and body weight. The test substance was applied once a day for 5 consecutive days starting from the very day of grouping. To prevent the rat from licking the foot, aluminum foil was wound around the external application site. About 5 hr after the final application, the volume of the both hind feet was measure by water substitution method, and difference from the measure at the time of grouping was calculated and evaluated.

The group constitution was as shown in the following Table 15B.

For a leflunomide suspended-type ointment, one prepared in Example was used, and for preparation blank, one prepared with the same composition but leflunomide was used.

TABLE 15B
Group constitution in evaluation test

| group | | | | number |
|---|---|---|---|---|
| No. | Test substance | Example | dose^{a)} | of animal |
| 1 | Blank ointment | — | 100 mg/foot | 6 |
| | preparation | | | |
| 2 | Leflunomide 1% | Example 23B | 100 mg/foot | 6 |
| | suspended-type | | | |
| | ointment b | | | |
| 3 | Leflunomide 5% | Example 22B | 100 mg/foot | 6 |
| | suspended-type | | | |
| | ointment a | | | |

^{a)}By one administration, 100 mg was applied to one foot and 200 mg to both feet.

The test results are shown in Table 16B.

TABLE 16B
Edema suppressive effect of leflunomide suspended ointment

| | | increase in hind foot | |
|---|---|---|---|
| | | volume^{a)} | |
| group | | right hind foot | left hind |
| No. | Test substance | (edema) | foot |
| 1 | Blank ointment preparation | 0.51 ± 0.32 | 0.35 ± 0.31 |
| 2 | Leflunomide 1% suspended-type | −0.07 ± 0.43 | −0.26 ± 0.28 |
| | ointment b | | |
| 3 | Leflunomide 5% suspended-type | −0.22 ± 0.25 | −0.31 ± 0.23 |
| | ointment a | | |

^{a)}unit amount of increase: ml, mean ± SD

As shown in the results, the leflunomide suspended ointment of the present invention showed an edema suppressive effect by the application thereof once a day.

### Industrial Applicability

A dosage form for external application containing SMP-114 or leflunomide or a pharmaceutically acceptable acid addition salt thereof of the present invention, and a soluble-type or suspended-type pharmaceutical composition for transdermal administration of the present invention of the present invention, which contains leflunomide or an active motabolite thereof or a pharmaceutically acceptable salt thereof, can show an effective antiinflammatory, antirheumatic effect by direct administration to the lesion or nearby skin.

This application is based on patent application Nos. 33975/2002, 219047/2002, 286418/2003 and 289457/2003 filed in Japan, the contents of which are hereby incorporated by reference.

1. A dosage form for external application comprising an isoxazole derivative having two substituents as an active ingredient.

2. The dosage form for external application of claim 1, wherein the isoxazole derivative is SMP-114 or a pharmaceutically acceptable acid addition salt thereof.

3. The dosage form for external application of claim 1, wherein the isoxazole derivative is leflunomide.

4. The dosage form for external application of claim 1, which is an ointment or cream.

5. The dosage form for external application of claim 1, which is a solution.

6. The dosage form for external application of claim 2, wherein an amount of SMP-114 is 0.1-10 w/w %.

7. The dosage form for external application of claims 2, wherein an amount of SMP-114 is 0.2-5 w/w %.

8. The dosage form for external application of claim 3, wherein an amount of leflunomide is 0.1-10 w/w %.

9. The dosage form for external application of claim 3, wherein an amount of leflunomide is 0.2-5 w/w %.

10. A pharmaceutical composition for transdermal administration, which comprises SMP-114 or a pharmaceutically acceptable acid addition salt thereof as an active ingredient.

11. A pharmaceutical composition for transdermal administration, which comprises a) leflunomide or an active motabolite thereof, or a pharmaceutically acceptable salt thereof as an active ingredient; and
b)(1) a carrier for transdermal administration, which comprises a base for dissolution in a proportion of not less than 40 w/w %, or
(2) a carrier for transdermal administration, which comprises a hydrophobic base for suspension, having no polar group in a molecule, in a proportion of not less than 70 w/w %.

12. The pharmaceutical composition of claim 11, wherein the active metabolite is N-(4-trifluoromethylpheyl)-2-cyano-3-hydroxy-crotonamide.

13. The pharmaceutical composition of claim 11, which is an active ingredient dissolution type composition, wherein the carrier for transdermal administration comprises a base for dissolution in a proportion of not less than 40 w/w %.

14. The pharmaceutical composition of claim 13, wherein the base for dissolution is one kind of base or a mixture of two or more kinds thereof selected from dibasic acid dialkyl esters, polyoxyethylene polyoxypropylene glycols, medium chain fatty acid triglycerides and macrogols.

15. The pharmaceutical composition of claim 13, wherein the carrier for transdermal administration has a hydrocarbon oil content of not more than 40 w/w %.

16. The pharmaceutical composition of claim 13, wherein the carrier for transdermal administration has a content of the base for dissolution of not less than 50 w/w %.

17. The pharmaceutical composition of claim 13, wherein the carrier for transdermal administration further comprises a lipophilic nonionic surfactant.

18. The pharmaceutical composition of claim 14, wherein the dibasic acid dialkyl ester is one kind of said ester or a mixture of two or more kinds thereof selected from diethyl sebacate, diisopropyl sebacate and diisopropyl adipate.

19. The pharmaceutical composition of claim 14, wherein the polyoxyethylene polyoxypropylene glycol is one kind of said glycol or a mixture of two or more kinds thereof selected from those that are liquid at 30° C.

20. The pharmaceutical composition of claim 14, wherein the medium chain fatty acid triglyceride is one kind of said triglyceride or a mixture of two or more kinds thereof selected from triglycerides comprising a fatty acid having 8 to 10 carbon atoms.

21. The pharmaceutical composition of claim 17, wherein the lipophilic nonionic surfactant is α-monoalkyl glyceryl ether.

22. The pharmaceutical composition of claim 17, wherein the carrier for transdermal administration has a lipophilic nonionic surfactant content of 0.1-10 w/w %.

23. The pharmaceutical composition of claim 13, which has a dosage form of a solution, an ointment, a gel preparation or a plaster.

24. The pharmaceutical composition of claim 13, which has a dosage form of a solution, an ointment or a gel preparation.

25. The pharmaceutical composition of claim 11, wherein the carrier for transdermal administration comprises a hydrophobic base for suspension, having no polar group in a molecule, in a proportion of not less than 70 w/w %, and the composition comprises the active ingredient stably suspended in the carrier.

26. The pharmaceutical composition of claim 25, wherein the hydrophobic base for suspension is a hydrocarbon oil.

27. The pharmaceutical composition of claim 25, wherein the hydrophobic base for suspension is a hydrocarbon gel.

28. The pharmaceutical composition of claim 25, wherein the carrier for transdermal administration consists only of a hydrophobic base for suspension having no polar group in a molecule.

29. The pharmaceutical composition of claim 25, wherein all the active ingredient particles suspended in the carrier for transdermal administration substantively have a particle size of not more than 100 μm and the average particle size is not more than 20 μm.

30. The pharmaceutical composition of claim 25, wherein all the active ingredient particles suspended in the carrier for transdermal administration substantively have a particle size of not more than 20 μm and the average particle size is not more than 10 μm.

31. The pharmaceutical composition of claim 25, which has a dosage form of an ointment, a liquid or a plaster.

32. The pharmaceutical composition of claim 25, which has a dosage form of a suspended ointment or a suspended liquid.

33. The pharmaceutical composition of claim 11, which comprises the active ingredient in a proportion of 0.1-10 w/w %.

34. Use of leflunomide or an active motabolite thereof, or a pharmaceutically acceptable salt thereof for the production of a transdermally administered therapeutic agent for chronic rheumatism or arthritis, which comprises a composition for transdermal administration of claim 1.

35. An administration method for the treatment of chronic rheumatism or arthritis, which comprises transdermally administering a pharmaceutical composition for transdermal administration, which comprises
a) leflunomide or an active motabolite thereof, or a pharmaceutically acceptable salt thereof as an active ingredient; and
b)(1) a carrier for transdermal administration, which comprises a base for dissolution in a proportion of not less than 40 w/w %, or
(2) a carrier for transdermal administration, which comprises a hydrophobic base for suspension having no polar group in a molecule in a proportion of not less than 70 w/w %.

36. The administration method of claim 35, wherein the carrier for transdermal administration comprises a base for dissolution in a proportion of not less than 40 w/w %, and the active ingredient is transdermally administered in a dissolution state in the carrier for transdermal administration.
